(19) United States
(12) Patent Application Publication
(10) Pub. No.: US 2007/0293657 A1
(43) Pub. Date: Dec. 20, 2007

(54) COMPLEXES AND METHODS OF FORMING COMPLEXES OF RIBONUCLEIC ACIDS AND PEPTIDES

(75) Inventors: Roger C. Adami, Snohomish, WA (US); Daniel Lyle Morris, Bellevue, WA (US); Henry R. Costantino, Woodinville, WA (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/670,905

(22) Filed: Feb. 2, 2007

Related U.S. Application Data
(60) Provisional application No. 60/774,852, filed on Feb. 17, 2006.

(51) Int. Cl. C07K 2/00; C07K 14/00; C07K 7/08

(52) U.S. Cl. 530/324; 530/300; 530/326; 530/327; 530/358

(57) ABSTRACT

A complex of a double stranded (ds) ribonucleic acid and a peptide produced by a method comprising dissolving the nucleic acid in an aqueous solution, dissolving the peptide in an aqueous solution, mixing the solubilized ds nucleic acid and the solubilized peptide, and treating the mixture by freezing and thawing, heating and cooling, or salting and desalting.

This application claims the benefit under 35 U.S.C. § 119(e) of U.S. Provisional Application No. 60/774,852, filed Feb. 17, 2006, which is hereby incorporated by reference in its entirety.

### BACKGROUND OF THE INVENTION

Delivering nucleic acids into animal and plant cells has long been an important object of molecular biology research and development. Recent developments in the areas of gene therapy, antisense therapy and RNA interference (RNAi) therapy have created a need to develop more efficient means for introducing nucleic acids into cells.

RNA interference is a process of sequence-specific post transcriptional gene silencing in cells initiated by a double-stranded (ds) polynucleotide, usually a dsRNA, that is homologous in sequence to a portion of a targeted messenger RNA (mRNA). Introduction of a suitable dsRNA into cells leads to destruction of endogenous, cognate mRNAs (i.e., mRNAs that share substantial sequence identity with the introduced dsRNA). The dsRNA molecules are cleaved by an RNase III family nuclease called dicer into short-interfering RNAs (siRNAs), which are 19-23 nucleotides (nt) in length. The siRNAs are then incorporated into a multicomponent nuclease complex known as the RNA-induced silencing complex or “RISC.” The RISC identifies mRNA substrates through their homology to the siRNA, and effectuates silencing of gene expression by binding to and destroying the targeted mRNA.

RNA interference is emerging a promising technology for modifying expression of specific genes in plant and animal cells, and is therefore expected to provide useful tools to treat a wide range of diseases and disorders amenable to treatment by modification of endogenous gene expression.

A variety of methods are available for delivering nucleic acid artificially into cells. These include transfection via calcium phosphate, cationic lipid, and lipsomal delivery. Nucleic acids can also be introduced into cells by electroporation and viral transduction. However, there are disadvantages to these methods. With viral gene delivery, there is a possibility that the replication deficient virus used as a delivery vehicle may revert to wild-type thus becoming pathogenic. Electroporation suffers from poor gene-transfer efficiency and therefore has limited clinical application. Finally, transfection may also be limited by poor efficiency and toxicity.

Synthetic and biological polypeptides show great potential as a tool to introduce nucleic acids into cells. However, synthetic peptides may elicit an undesired immune response and may be toxic because it is not be readily susceptible to degradation in the cell.

Biological peptides, i.e., fragments of naturally occurring proteins, typically do not suffer from the same disadvantages as synthetic peptides. Nonetheless, both biological and synthetic peptides can suffer from non-specific promiscuous aggregation when complexed with nucleic acids at physiological salt concentrations. Consequently, this instability severely limits the effectiveness of delivery of the nucleic acid via the polypeptide. Therefore, there remains a need for improved methods and formulations to deliver siNAs in an effective amount, in an active and enduring state, and using non-toxic delivery vehicles, to selected cells, tissues, or compartments to mediate regulation of gene expression in a manner that will alter a phenotype or disease state of the targeted cells.

### BRIEF SUMMARY OF THE INVENTION

One aspect of the invention is a complex between a double stranded (ds) nucleic acid and a peptide produced by a method comprising:

(a) dissolving/solubilizing the nucleic acid in an aqueous solution;

(b) dissolving the peptide in an aqueous solution;

(c) mixing the dissolved ds nucleic acid and the solubilized peptide; and

(d) treating the mixture by freezing and thawing.

Another aspect of the invention is a complex between a double stranded (ds) nucleic acid and a peptide produced by a method comprising:

(a) solubilizing the nucleic acid in an aqueous solution;

(b) solubilizing the peptide in an aqueous solution;

(c) mixing the solubilized ds nucleic acid and the solubilized peptide; and

(d) treating the mixture by heating and cooling.

Yet another aspect of the invention is a complex between a double stranded (ds) nucleic acid and a peptide produced by a method comprising:

(a) Solubilizing the nucleic acid in an aqueous solution;

(b) solubilizing the peptide in an aqueous solution;

(c) mixing the solubilized ds nucleic acid and the solubilized peptide; and

(d) treating the mixture by raising the salt concentration, and dialyzing to remove the salt.

In some embodiments, the ds nucleic acid is a dsRNA. In some embodiments, the dsRNA is a siRNA having 29-50 base pairs. In some embodiments, the siRNA contains a sequence that is complementary to a region of a TNF-alpha gene. In some embodiments, the ds nucleic acid is a dsDNA. In some embodiments, the peptide is a polynucleotide delivery-enhancing polypeptide, which may contain a histone protein, or a polypeptide or peptide fragment, derivative, analog, or conjugate thereof. In some embodiments, the polynucleotide delivery-enhancing polypeptide may include an amphipathic amino acid sequence. In some embodiments, the polynucleotide delivery-enhancing polypeptide contains a protein transduction domain or motif. In some embodiments, the polynucleotide delivery-enhancing polypeptide contains a fusogenic peptide domain or motif. In some embodiments, the polynucleotide delivery-enhancing polypeptide comprises a nucleic acid-binding domain or motif. In some embodiments, the peptide binds a ds nucleic acid with a Kd less than about 100 nM, or less than about 10 nM. In some embodiments, the polynucleotide delivery-enhancing polypeptide may be selected from the group consisting of:

(SEQ ID NO: 34)

GRKKRRQRRRPPQC

(SEQ ID NO: 35)

Maleimide-AAVALLPAVLLALLAPRKKRRQRRRPPQ-amide

(SEQ ID NO: 36)

AAVALLPAVLLALLAPRKKRRQRRRPPQC

(SEQ ID NO: 37)

Maleimide-AAVALLPAVLLALLAPRKKRRQRRRPPQ-amide

(SEQ ID NO: 38)

NH2-RKKRRQRRRPPQCAAVALLPAVLLALLAP-amide

(SEQ ID NO: 39)

BrAc-GRKKRRQRRRPQ-amide

(SEQ ID NO: 40)

BrAc-RRRQRRKRGGDIMGEWGNEIFGAIAGFLG-amide

(SEQ ID NO: 41)

NH2-RRRQRRKRGGDIMGEWGNEIFGAIAGFLG-amide

(SEQ ID NO: 42)

CYGRKKRRQRRRGYGRKKRRQRRRG

(SEQ ID NO: 43)

Maleimide-GRKKRRQRRRPPQ-amide

(SEQ ID NO: 44)

NH2-KLWKAWPKLWKKLWKP-amide

(SEQ ID NO: 45)

AAVALLPAVLLALLAPRRRRRR-amide

(SEQ ID NO: 46)

RLWRALPRVLRRLLRP-amide

(SEQ ID NO: 47)

NH2-AAVALLPAVLLALLAPSGASGLDKRDYV-amide

(SEQ ID NO: 48)

Maleimide-AAVALLPAVLLALLAPSGASGLDKRDYV-amide

(SEQ ID NO: 49)

NH2-SGASGLDKRDYVAAVAALLPAVLLALLAP-amide

(SEQ ID NO: 50)

NH2-LLETLLKPFQCRICMRNFSTRQARRNHRRRHRR-amide

(SEQ ID NO: 51)

NH2-AAVACRICMRNFSTRQARRNHRRRHRR-amide

(SEQ ID NO: 52)

Maleimide-RQIKIWFQNRRMKWKK-amide

(SEQ ID NO: 53)

RQIKIWFQNRRMKWKK-amide

(SEQ ID NO: 54)

NH2-RQIKIWFQNRRMKWKKDIMGEWGNEIFGAIAGFLG-amide

(SEQ ID NO: 55)

Maleimide-SGRGKQGGKARAKAKTRSSRAGLQFPVGRVHRLLRKG-
amide

(SEQ ID NO: 56)

SGRGKQGGKARAKAKTRSSRAGLQFPVGRVHRLLRKGC-amide

(SEQ ID NO: 57)

KGSKKAVTKAQKKDGKKRKRSRK-amide

(SEQ ID NO: 58)

NH2-KKDGKKRKRSRKESYSVYVYKVLKQ-amide

(SEQ ID NO: 59)

KGSKKAVTKAQKKDGKKRKRSRKESYSVYVYKVLKQ

(SEQ ID NO: 60)

BrAc-GWTLNSAGYLLGKINLKALAALAKKIL-amide

(SEQ ID NO: 61)

KLALKLALKALKAALKLA-amide

(SEQ ID NO: 62)

BrAc-KLALKLALKALKAALKLA-amide

(SEQ ID NO: 63)

Ac-KETWWETWWTEWSQPKKKRKV-amide

(SEQ ID NO: 64)

NH2-KETWWETWWTEWSQPGRKKRRQRRRPPQ-amide

(SEQ ID NO: 65)

BrAc-RRRRRRR

(SEQ ID NO: 66)

QqQqQqQqQq

(SEQ ID NO: 67)

NH2-RRRQRRKRGGqQqQqQqQqQ-amide

(SEQ ID NO: 68)

RVIRWFQNKRCKDKK-amide

(SEQ ID NO: 69)

Ac-LGLLLRHLRHHSNLLANI-amide

(SEQ ID NO: 70)

GQMSEIEAKVRTVKLARS-amide

(SEQ ID NO: 71)

NH2-KLWSAWPSLWSSLWKP-amide

(SEQ ID NO: 72)

NH2-KKKKKKKKK-amide

(SEQ ID NO: 73)

NH2-AARLHRFKNKGKDSTEMRRRR-amide

(SEQ ID NO: 74)

Maleimide-GLGSLLKKAGKKLKQPKSKRKV-amide

(SEQ ID NO: 75)

Maleimide-Dmt-r-FK-amide
(Dmt is dimethyltyrosine, r is D-Arg)

(SEQ ID NO: 76)

Maleimide-Dmt-r-FKQqQqQqQqQq-amide

(SEQ ID NO: 77)

Maleimide-WRFK-amide

(SEQ ID NO: 78)

Maleimide-WRFKQqQqQqQqQq-amide

(SEQ ID NO: 79)

Maleimido-YRFK-amide

(SEQ ID NO: 80)

Maleimide-YRFKYRFKYRFK-amide

(SEQ ID NO: 81)

Maleimide-WRFK-amide

(SEQ ID NO: 82)

Maleimide-WRFKKSKRKV-amide

(SEQ ID NO: 83)

Maleimide-WRFKAAVALLPAVLLALLAP-amide

(SEQ ID NO: 84)

NH2-DiMeYrFK-amide (DiMeY is mimethyltyrosine)

(SEQ ID NO: 85)

NH2-YrFK-amide

(SEQ ID NO: 86)

NH2-DiMeYRFK-amide

(SEQ ID NO: 87)

NH2-WrFK-amide

(SEQ ID NO: 88)

NH2-DiMeYrWK-amide

(SEQ ID NO: 89)

NH2-KFrDiMeY-amide

(SEQ ID NO: 90)

Maleimide-WRFKWRFK-amide
and

(SEQ ID NO: 91)

Maleimide-WRFKWRFKWRFK-amide

In some embodiments, the polynucleotide delivery-enhancing polypeptide may be one or more peptides selected from histone H1, histone H₂B, histone H3, histone H4, a histone fragment thereof,

(SEQ ID NO: 92)

GKINLKALAALAKKIL,

(SEQ ID NO: 93)

RVIRVWFQNKRCKDKK,

(SEQ ID NO: 94)

GRKKRRQRRRPPQGRKKRRQRRRPPQGRKKRRQRRRPPQ,

(SEQ ID NO: 95)

GEQIAQLIAGYIDIILKKKKSK,

(SEQ ID NO: 96)

WWETWKPFQCRICMRNFSTRQARRNHRRRHR,
Poly Lys-Trp (4:1, MW 20,000-50,000),
Poly Orn-Trp (4:1, MW 20,000-50,000),
and
mellitin.

In some embodiments, the delivery-enhancing polypeptide is PN73 having the structure:

(SEQ ID NO: 100)

KGSKKAVTKAQKKDGKKRKRSRKESYSVYVYKVLKQ.

### DETAILED DESCRIPTION OF THE INVENTION

This invention describes methods to form siRNA/polypeptide complexes that improve the gene expression knockdown activity mediated by the siRNA molecule. The various methods used to structure the polypeptide and siRNA are as follows: (1) dialysis from various salts or peptide denaturants; (2) heating and cooling cycles; (3) freeze-thawing, and (4) pH titration. These processes affect the interactions of the polypeptide and siRNA in a manner that leads to increased transfection efficacy. These changes are driven by the addition of an external agent or energy that enables favorable interactions between the polypeptide and siRNA molecule creating an “optimized” complex that remains stable upon removal of the external agent or energy from the system. In general, these methods of treatment may be regarded as an “annealing” process.

A surprising and unexpected discovery of the present invention was improved gene knockdown activity of approximately 19% over that of non-treated siRNA/polypeptide complexes (based on averages for the various peptides and different siRNA/polypeptide ratios). This degree of improvement was noted for both the freeze-thaw method and heating-cooling method. This improvement may be further enhanced by the addition of other agents to the formulation.

This invention provides novel compositions and methods that employ a short interfering nucleic acid (siNA), or a precursor thereof, in combination with a polynucleotide delivery-enhancing polypeptide and an organic counter-ion. The polynucleotide delivery-enhancing polypeptide is a natural or artificial polypeptide selected for its ability to enhance intracellular delivery or uptake of polynucleotides, including siNAs and their precursors. The counter-ion is an organic acid or base that stabilizes the siNA and polynucleotide delivery-enhancing polypeptide complex in solution.

The compositions and methods of the invention are useful as therapeutic tools to regulate expression of tumor necrosis factor-alpha (TNF-α) to treat or prevent symptoms of rheumatoid arthritis (RA). In this context the invention further provides compounds, compositions, and methods useful for modulating expression and activity of TNF-α by RNA interference (RNAi) using the short interfering RNA molecule LC20. LC20 is a double stranded 21-mer siRNA molecule with sequence homology to the human TNF-α gene. The LC20 nucleotide sequence is as follows:

(SEQ ID NO: 32)

GGGUCGGAACCCAAGCUUATT

(SEQ ID NO: 33)

ATCCCAGCCUUGGGUUCGAAU

In some embodiments, this invention provides a short interfering nucleic acid (siNA), a short interfering RNA (siRNA), a double-stranded RNA (dsRNA), a micro-RNA (mRNA), or a short hairpin RNA (shRNA) molecule, and methods of preparing complexes of these molecules that are effective for modulating expression of TNF-α and/or TNF-α genes, which can be applied to prevent or alleviate symptoms of RA in mammalian subjects, as well as other (TNF-α)-associated diseases. Within these and related therapeutic compositions and methods, the use of chemically-modified siNAs will often improve properties of the modified siNAs in comparison to properties of native siNA molecules, for example by providing increased resistance to nuclease degradation in vivo, and/or through improved cellular uptake. As can be readily determined according to the disclosure herein, useful siNAs having multiple chemical modifications will retain their RNAi activity. The siNA molecules of the instant invention thus provide useful reagents and methods for a variety of therapeutic, diagnostic, target validation, genomic discovery, genetic engineering, and pharmacogenomic applications.

Administration

This siNAs of the present invention may be administered in any form, for example transdermally or by local injection (e.g., local injection at sites of psoriatic plaques to treat psoriasis, or into the joints of patients afflicted with psoriatic arthritis or RA). In more detailed embodiments, the invention provides formulations and methods to administer therapeutically effective amounts of siNAs directed against of a mRNA of TNF-α, which effectively down-regulate the TNF-α RNA and thereby reduce or prevent one or more TNF-α-associated inflammatory condition(s). Comparable methods and compositions are provided that target expression of one or more different genes associated with a selected disease condition in animal subjects, including any of a large number of genes whose expression is known to be aberrantly increased as a causal or contributing factor associated with the selected disease condition.

The siNA/polynucleotide delivery-enhancing polypeptide mixtures of the invention can be administered in conjunction with other standard treatments for a targeted disease condition, for example in conjunction with therapeutic agents effective against inflammatory diseases, such as RA or psoriasis. Examples of combinatorially useful and effective agents in this context include non-steroidal antiinflammatory drugs (NSAIDs), methotrexate, gold compounds, D-penicillamine, the antimalarials, sulfasalazine, glucocorticoids, and other TNF-α neutralizing agents such as infliximab and entracept.

Negatively charged polynucleotides of the invention (e.g., RNA or DNA) can be administered to a patient by any standard means, with or without stabilizers or buffers, to form a pharmaceutical composition. When it is desired to use a liposome delivery mechanism, standard protocols for formation of liposomes can be followed. The compositions of the present invention may also be formulated and used as tablets, capsules or elixirs for oral administration, suppositories for rectal administration, sterile solutions, suspensions for injectable administration, and the other compositions known in the art.

The present invention also includes pharmaceutically acceptable formulations of the compositions described herein. These formulations include salts of the above compounds, e.g., acid addition salts, for example, salts of hydrochloric, hydrobromic, acetic acid, and benzene sulfonic acid.

A pharmacological composition or formulation refers to a composition or formulation in a form suitable for administration, e.g., systemic administration, into a cell or patient, including for example a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmacological compositions injected into the blood stream should be soluble. Other factors are known in the art, and include considerations such as toxicity.

In exemplary embodiments, the instant invention features compositions comprising a small nucleic acid molecule, such as short interfering nucleic acid (siNA), a short interfering RNA (siRNA), a double-stranded RNA (dsRNA), micro-RNA (mRNA), or a short hairpin RNA (shRNA), admixed or complexed with, or conjugated to, a polynucleotide delivery-enhancing polypeptide.

As used herein, the term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule”, refers to any nucleic acid molecule capable of inhibiting or down regulating gene expression or viral replication, for example by mediating RNA interference “RNAi” or gene silencing in a sequence-specific manner. Within exemplary embodiments, the siNA is a double-stranded polynucleotide molecule comprising self-complementary sense and antisense regions, wherein the antisense region comprises a nucleotide sequence that is complementary to a nucleotide sequence in a target nucleic acid molecule for down regulating expression, or a portion thereof, and the sense region comprises a nucleotide sequence corresponding to (i.e., which is substantially identical in sequence to) the target nucleic acid sequence or portion thereof.

“siNA” means a small interfering nucleic acid, for example a siRNA, that is a short-length double-stranded nucleic acid (or optionally a longer precursor thereof), and which is not unacceptably toxic in target cells. The length of useful siNAs within the invention will in certain embodiments be optimized at a length of approximately 20 to 50 bp long. However, there is no particular limitation in the length of useful siNAs, including siRNAs. For example, siNAs can initially be presented to cells in a precursor form that is substantially different than a final or processed form of the siNA that will exist and exert gene silencing activity upon delivery, or after delivery, to the target cell. Precursor forms of siNAs may, for example, include precursor sequence elements that are processed, degraded, altered, or cleaved at or following the time of delivery to yield a siNA that is active within the cell to mediate gene silencing. Thus, in certain embodiments, useful siNAs within the invention will have a precursor length, for example, of approximately 100-200 base pairs, 50-100 base pairs, or less than about 50 base pairs, which will yield an active, processed siNA within the target cell. In other embodiments, a useful siNA or siNA precursor will be approximately 10 to 49 bp, 15 to 35 bp, or about 21 to 30 bp in length.

In certain embodiments of the invention, as noted above, polynucleotide delivery-enhancing polypeptides are used to facilitate delivery of larger nucleic acid molecules than conventional siNAs, including large nucleic acid precursors of siNAs. For example, the methods and compositions herein may be employed for enhancing delivery of larger nucleic acids that represent “precursors” to desired siNAs, wherein the precursor amino acids may be cleaved or otherwise processed before, during or after delivery to a target cell to form an active siNA for modulating gene expression within the target cell. For example, a siNA precursor polynucleotide may be selected as a circular, single-stranded polynucleotide, having two or more loop structures and a stem comprising self-complementary sense and antisense regions, wherein the antisense region comprises a nucleotide sequence that is complementary to a nucleotide sequence in a target nucleic acid molecule or a portion thereof, and the sense region having nucleotide sequence corresponding to the target nucleic acid sequence or a portion thereof, and wherein the circular polynucleotide can be processed either in vivo or in vitro to generate an active siNA molecule capable of mediating RNAi.

In mammalian cells, dsRNAs longer than 30 base pairs can activate the dsRNA-dependent kinase PKR and 2′-5′-oligoadenylate synthetase, normally induced by interferon. The activated PKR inhibits general translation by phosphorylation of the translation factor eukaryotic initiation factor 2α (eIF2α), while 2′-5′-oligoadenylate synthetase causes nonspecific mRNA degradation via activation of RNase L. By virtue of their small size (referring particularly to non-precursor forms), usually less than 30 base pairs, and most commonly between about 17-19, 19-21, or 21-23 base pairs, the siNAs of the present invention avoid activation of the interferon response.

In contrast to the nonspecific effect of long dsRNA, siRNA can mediate selective gene silencing in the mammalian system. Hairpin RNAs, with a short loop and 19 to 27 base pairs in the stem, also selectively silence expression of genes that are homologous to the sequence in the double-stranded stem. Mammalian cells can convert short hairpin RNA into siRNA to mediate selective gene silencing.

RISC mediates cleavage of single stranded RNA having sequence complementary to the antisense strand of the siRNA duplex. Cleavage of the target RNA takes place in the middle of the region complementary to the antisense strand of the siRNA duplex. Studies have shown that 21 nucleotide siRNA duplexes are most active when containing two nucleotide 3′-overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) has been reported to be tolerated.

Studies have shown that replacing the 3′-overhanging segments of a 21-mer siRNA duplex having 2 nucleotide 3′ overhangs with deoxyribonucleotides does not have an adverse effect on RNAi activity. Replacing up to 4 nucleotides on each end of the siRNA with deoxyribonucleotides has been reported to be well tolerated whereas complete substitution with deoxyribonucleotides results in no RNAi activity.

Alternatively, the siNAs can be delivered as single or multiple transcription products expressed by a polynucleotide vector encoding the single or multiple siNAs and directing their expression within target cells. In these embodiments the double-stranded portion of a final transcription product of the siRNAs to be expressed within the target cell can be, for example, 15 to 49 bp, 15 to 35 bp, or about 21 to 30 bp long. Within exemplary embodiments, double-stranded portions of siNAs, in which two strands pair up, are not limited to completely paired nucleotide segments, and may contain nonpairing portions due to mismatch (the corresponding nucleotides are not complementary), bulge (lacking in the corresponding complementary nucleotide on one strand), overhang, and the like. Nonpairing portions can be contained to the extent that they do not interfere with siNA formation. In more detailed embodiments, a “bulge” may comprise 1 to 2 nonpairing nucleotides, and the double-stranded region of siNAs in which two strands pair up may contain from about 1 to 7, or about 1 to 5 bulges. In addition, “mismatch” portions contained in the double-stranded region of siNAs may be present in numbers from about 1 to 7, or about 1 to 5. Most often in the case of mismatches, one of the nucleotides is guanine, and the other is uracil. Such mismatching may be attributable, for example, to a mutation from C to T, G to A, or mixtures thereof, in a corresponding DNA coding for sense RNA, but other cause are also contemplated. Furthermore, in the present invention the double-stranded region of siNAs in which two strands pair up may contain both bulge and mismatched portions in the approximate numerical ranges specified.

The terminal structure of siNAs of the invention may be either blunt or cohesive (overhanging) as long as the siNA retains its activity to silence expression of target genes. The cohesive (overhanging) end structure is not limited only to the 3′ overhang as reported by others. On the contrary, the 5′ overhanging structure may be included as long as it is capable of inducing a gene silencing effect such as by RNAi. In addition, the number of overhanging nucleotides is not limited to reported limits of 2 or 3 nucleotides, but can be any number as long as the overhang does not impair gene silencing activity of the siNA. For example, overhangs may comprise from about 1 to 8 nucleotides, more often from about 2 to 4 nucleotides. The total length of siNAs having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp. Furthermore, since the overhanging sequence may have low specificity to a target gene, it is not necessarily complementary (antisense) or identical (sense) to the target gene sequence. Furthermore, as long as the siNA is able to maintain its gene silencing effect on the target gene, it may contain low molecular weight structure (for example a natural RNA molecule such as tRNA, rRNA or viral RNA, or an artificial RNA molecule), for example, in the overhanging portion at one end.

In addition, the terminal structure of the siNAs may have a stem-loop structure in which ends of one side of the double-stranded nucleic acid are connected by a linker nucleic acid, e.g., a linker RNA. The length of the double-stranded region (stem-loop portion) can be, for example, 15 to 49 bp, often 15 to 35 bp, and more commonly about 21 to 30 bp long. Alternatively, the length of the double-stranded region that is a final transcription product of siNAs to be expressed in a target cell may be, for example, approximately 15 to 49 bp, 15 to 35 bp, or about 21 to 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop siRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example, Martinez, et al., Cell 110:563-574, 2002, and Schwarz, et al., Molecular Cell 10:537-568, 2002, or 5′,3′-diphosphate.

As used herein, the term siNA molecule is not limited to molecules containing only naturally-occurring RNA or DNA, but also encompasses chemically-modified nucleotides and non-nucleotides. In certain embodiments, the short interfering nucleic acid molecules of the invention lack 2′-hydroxy (2′-OH) containing nucleotides. In certain embodiments short interfering nucleic acids do not require the presence of c acid molecules of the invention optionally do not include any ribonucleotides (e.g., nucleotides having a 2′-hydroxy group for mediating RNAi and as such, short interfering nucleotides having a 2′-OH group). Such siNA molecules that do not require the presence of ribonucleotides within the siNA molecule to support RNAi can however have an attached linker or linkers or other attached or associated groups, moieties, or chains containing one or more nucleotides with 2′-OH groups. Optionally, siNA molecules can comprise ribonucleotides at about 5, 10, 20, 30, 40, or 50% of the nucleotide positions.

As used herein, the term siNA is meant to be equivalent to other terms used to describe nucleic acid molecules that are capable of mediating sequence specific RNAi, for example short interfering RNA (siRNA), double-stranded RNA (dsRNA), micro-RNA (mRNA), short hairpin RNA (shRNA), short interfering oligonucleotide, short interfering nucleic acid, short interfering modified oligonucleotide, chemically-modified siRNA, post-transcriptional gene silencing RNA (ptgsRNA), and others.

In other embodiments, siNA molecules for use within the invention may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic intercations, and/or stacking interactions.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. These antisense and sense RNAs have been conventionally synthesized with an RNA synthesizer.

As used herein, the term “RNAi construct” is a generic term used throughout the specification to include small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo. Optionally, the siRNA include single strands or double strands of siRNA.

A siHybrid molecule is a double-stranded nucleic acid that has a similar function to siRNA. Instead of a double-stranded RNA molecule, a siHybrid is comprised of an RNA strand and a DNA strand. Preferably, the RNA strand is the antisense strand as that is the strand that binds to the target mRNA. The siHybrid created by the hybridization of the DNA and RNA strands have a hybridized complementary portion and preferably at least one 3′ overhanging end.

siNAs for use within the invention can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, for example wherein the double stranded region is about 19 base pairs). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the siNA can be assembled from a single oligonucleotide, where the self-complementary sense and antisense regions of the siNA are linked by means of a nucleic acid-based or non-nucleic acid-based linker(s).

Within additional embodiments, siNAs for intracellular delivery according to the methods and compositions of the invention can be a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that can be made in an siNA include without limitation phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in various siNA constructs, are shown to preserve RNAi activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically-modified siNA can also minimize the possibility of activating interferon activity in humans.

The siNA molecules described herein, the antisense region of a siNA molecule of the invention can comprise a phosphorothioate internucleotide linkage at the 3′-end of said antisense region. In any of the embodiments of siNA molecules described herein, the antisense region can comprise about one to about five phosphorothioate internucleotide linkages at the 5′-end of said antisense region. In any of the embodiments of siNA molecules described herein, the 3′-terminal nucleotide overhangs of a siNA molecule of the invention can comprise ribonucleotides or deoxyribonucleotides that are chemically-modified at a nucleic acid sugar, base, or backbone. In any of the embodiments of siNA molecules described herein, the 3′-terminal nucleotide overhangs can comprise one or more universal base ribonucleotides. In any of the embodiments of siNA molecules described herein, the 3′-terminal nucleotide overhangs can comprise one or more acyclic nucleotides.

For example, in a non-limiting example, the invention features a chemically-modified short interfering nucleic acid (siNA) having about 1, 2, 3, 4, 5, 6, 7, 8 or more phosphorothioate internucleotide linkages in one siNA strand. In yet another embodiment, the invention features a chemically-modified short interfering nucleic acid (siNA) individually having about 1, 2, 3, 4, 5, 6, 7, 8 or more phosphorothioate internucleotide linkages in both siNA strands. The phosphorothioate internucleotide linkages can be present in one or both oligonucleotide strands of the siNA duplex, for example in the sense strand, the antisense strand, or both strands. The siNA molecules of the invention can comprise one or more phosphorothioate internucleotide linkages at the 3′-end, the 5′-end, or both of the 3′- and 5′-ends of the sense strand, the antisense strand, or both strands. For example, an exemplary siNA molecule of the invention can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, an exemplary siNA molecule of the invention can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, an exemplary siNA molecule of the invention can comprise one or more (e.g., about 1, 2, 3, 4, 5, 6, 7, 8, 9, 10, or more) purine phosphorothioate internucleotide linkages in the sense strand, the antisense strand, or both strands.

An siNA molecule may be comprised of a circular nucleic acid molecule, wherein the siNA is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular siNA molecule contains two loop motifs, wherein one or both loop portions of the siNA molecule is biodegradable. For example, a circular siNA molecule of the invention is designed such that degradation of the loop portions of the siNA molecule in vivo can generate a double-stranded siNA molecule with 3′-terminal overhangs, such as 3′-terminal nucleotide overhangs comprising about 2 nucleotides.

Modified nucleotides present in siNA molecules, preferably in the antisense strand of the siNA molecules, but also optionally in the sense and/or both antisense and sense strands, comprise modified nucleotides having properties or characteristics similar to naturally occurring ribonucleotides. For example, the invention features siNA molecules including modified nucleotides having a Northern conformation (e.g., Northern pseudorotation cycle, see for example, Saenger, Principles of Nucleic Acid Structure, Springer-Verlag ed., 1984). As such, chemically modified nucleotides present in the siNA molecules of the invention, preferably in the antisense strand of the siNA molecules of the invention, but also optionally in the sense and/or both antisense and sense strands, are resistant to nuclease degradation while at the same time maintaining the capacity to mediate RNAi. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro micleotides. 2′-deoxy-2′-chloro nucleotides, 2′-azido nucleotides, and 2′-O-methyl nucleotides.

The sense strand of a double stranded siNA molecule may have a terminal cap moiety such as an inverted deoxyabasic moiety, at the 3′-end, 5′-end, or both 3′ and 5′-ends of the sense strand.

Non-limiting examples of conjugates include conjugates and ligands described in Vargeese, et al., U.S. application Ser. No. 10/427,160, filed Apr. 30, 2003, incorporated by reference herein in its entirety, including the drawings. In another embodiment, the conjugate is covalently attached to the chemically-modified siNA molecule via a biodegradable linker. In one embodiment, the conjugate molecule is attached at the 3′-end of either the sense strand, the antisense strand, or both strands of the chemically-modified siNA molecule. In another embodiment, the conjugate molecule is attached at the 5′-end of either the sense strand, the antisense strand, or both strands of the chemically-modified siNA molecule. In yet another embodiment, the conjugate molecule is attached both the 3′-end and 5′-end of either the sense strand, the antisense strand, or both strands of the chemically-modified siNA molecule, or any combination thereof. In one embodiment, a conjugate molecule of the invention comprises a molecule that facilitates delivery of a chemically-modified siNA molecule into a biological system, such as a cell. In another embodiment, the conjugate molecule attached to the chemically-modified siNA molecule is a poly ethylene glycol, human serum albumin, or a ligand for a cellular receptor that can mediate cellular uptake. Examples of specific conjugate molecules contemplated by the instant invention that can be attached to chemically-modified siNA molecules are described in Vargeese, et al., U.S. Patent Application Publication No. 20030130186, published Jul. 10, 2003, and U.S. Patent Application Publication No. 20040110296, published Jun. 10, 2004. The type of conjugates used and the extent of conjugation of siNA molecules of the invention can be evaluated for improved pharmacokinetic profiles, bioavailability, and/or stability of siNA constructs while at the same time maintaining the ability of the siNA to mediate RNAi activity. As such, one skilled in the art can screen siNA constructs that are modified with various conjugates to determine whether the siNA conjugate complex possesses improved properties while maintaining the ability to mediate RNAi, for example in animal models as are generally known in the art.

A siNA further may be further comprised of a nucleotide, non-nucleotide, or mixed nucleotide/non-nucleotide linker that joins the sense region of the siNA to the antisense region of the siNA. In one embodiment, a nucleotide linker can be a linker of >2 nucleotides in length, for example about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art. [See, for example, Gold, et al, Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma, et al., Nucleic Acids Res. 21:2585, 1993, and Biochemistry 32:1751, 1993; Durand, et al., Nucleic Acids Res. 18:6353, 1990; McCurdy, et al., Nucleosides & Nucleotides 10:287, 1991; Jschke, et al., Tetrahedron Lett. 34:301, 1993; Ono, et al., Biochemistry 30:9914, 1991; Arnold, et al., International Publication No. WO 89/02439; Usman, et al., International Publication No. WO 95/06731; Dudycz, et al., International Publication No. WO 95/11910, and Ferentz and Verdine, J. Am. Chem. Soc. 113:4000, 1991. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thyrnine, for example at the C1 position of the sugar.

In one embodiment, the invention features modified siNA molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, Nucleic Acid Analogues: Synthesis and Properties, in Modern Synthetic Methods, VCH, 331-417, 1995, and Mesmaeker, et al., Novel Backbone Replacements for Oligonucleotides, in Carbohydrate Modifications in Antisense Research, ACS, 24-39, 1994.

Synthesis of siNA

The synthesis of a siNA molecule of the invention, which can be chemically-modified, comprises: (a) synthesis of two complementary strands of the siNA molecule; (b) annealing the two complementary strands together under conditions suitable to obtain a double-stranded siNA molecule.

In some embodiments, synthesis of the two complementary strands of the siNA molecule is by solid phase oligonucleotide synthesis. In some embodiments, synthesis of the two complementary strands of the siNA molecule is by solid phase tandem oligonucleotide synthesis.

Oligonucleotides (e.g., certain modified oligonucleotides or portions of oligonucleotides lacking ribonucleotides) are synthesized using protocols known in the art, for example as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., 1997, Methods Mol. Bio. 74:59, 1997; Brennan, et al., Biotechnol Bioeng. 61:33-45, 1998, and Brennan, U.S. Pat. No. 6,001,311. Synthesis of RNA, including certain siNA molecules of the invention, follows general procedures as described, for example, in Usman, et al., 1987, J. Am. Chem. Soc. 109:7845, 1987; Scaringe, et al., Nucleic Acids Res. 18:5433, 1990; and Wincott, et al., Nucleic Acids Res. 23:2677-2684, 1995; Wincott, et al., Methods Mol. Bio. 74:59, 1997.

Supplemental or complementary methods for delivery of nucleic acid molecules for use within then invention are described, for example, in Akhtar, et al., Trends Cell Bio. 2:139, 1992; Delivery Strategiesfor Antisense Oligonucleotide Therapeutics, ed. Akhtar, 1995, Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No WO 94/02595, further describes general methods for delivery of enzymatic nucleic acid molecules. These protocols can be utilized to supplement or complement delivery of virtually any nucleic acid molecule contemplated within the invention.

Delivery Methods

Nucleic acid molecules and polynucleotide delivery-enhancing polypeptides can be administered to cells by a variety of methods known to those of skill in the art, including, but not restricted to, administration within formulations that comprise the siNA and polynucleotide delivery-enhancing polypeptide alone, or that further comprise one or more additional components, such as a pharmaceutically acceptable carrier, diluent, excipient, adjuvant, emulsifier, buffer, stabilizer, preservative, and the like. In certain embodiments, the siNA and/or the polynucleotide delivery-enhancing polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see e.g., O'Hare and Normand, International PCT Publication No. WO 00/53722). Alternatively, a nucleic acid/peptide/vehicle combination can be locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262.

Methods for the delivery of nucleic acid molecules are described in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Beigelman, et al., U.S. Pat. No. 6,395,713 and Sullivan, et al., PCT WO 94/02595 further describe the general methods for delivery of nucleic acid molecules. These protocols can be utilized for the delivery of virtually any nucleic acid molecule. Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example, Gonzalez, et al., Bioconjugate Chem. 10: 1068-1074, 1999; Wang, et al., International PCT publication Nos. WO 03/47518 and WO 03/46185), poly(lactic-co-glycolic)ac-id (PLGA) and PLCA microspheres (see for example, U.S. Pat. No. 6,447,796 and U.S. Patent Application Publication No. US 2002130430), biodegradable nanocapsules, and bioadhesive microspheres, or by proteinaceous vectors (O'Hare and Normand, International PCT Publication No. WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry, et al., Clin. Cancer Res. 5:2330-2337, 1999, and Barry, et al., International PCT Publication No. WO 99/31262. The molecules of the instant invention can be used as pharmaceutical agents. Pharmaceutical agents prevent, modulate the occurrence, or treat (alleviate a symptom to some extent, preferably all of the symptoms) of a disease state in a subject.

Within the compositions, formulations and methods of this invention, the active agent may be combined or coordinately administered with a suitable carrier or vehicle. As used herein, the term “carrier” means a pharmaceutically acceptable solid or liquid filler, diluent or encapsulating or carrying material.

A carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. Examples of ingredients, pharmaceutical excipients and/or additives of the above categories suitable for use in the compositions and formulations of this invention can be found in the U.S. Pharmacopeia National Formulary, 1990, pp. 1857-1859, as well as in Raymond C. Rowe, et al., Handbook of Pharmaceutical Excipients, 5th ed., 2006, and Remington: The Science and Practice of Pharmacy, 21st ed., 2006, editor David B. Troy, and in the Physician's Desk Reference, 52nd ed., Medical Economics, Montvale, N.J., 1998.

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other non toxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

The term “ligand” refers to any compound or molecule, such as a drug, peptide, hormone, or neurotransmitter that is capable of interacting with another compound, such as a receptor, either directly or indirectly. The receptor that interacts with a ligand can be present on the surface of a cell or can alternately be an intercellular receptor. Interaction of the ligand with the receptor can result in a biochemical reaction, or can simply be a physical interaction or association.

By “asymmetric hairpin” as used herein is meant a linear siNA molecule comprising an antisense region, a loop portion that can comprise nucleotides or non-nucleotides, and a sense region that comprises fewer nucleotides than the antisense region to the extent that the sense region has enough complementary nucleotides to base pair with the antisense region and form a duplex with loop. For example, an asymmetric hairpin siNA molecule of the invention can comprise an antisense region having length sufficient to mediate RNAi in a T-cell (e.g., about 19 to about 22 (e.g., about 19, 20, 21, or 22) nucleotides) and a loop region comprising about 4 to about 8 (e.g., about 4, 5, 6, 7, or 8) nucleotides, and a sense region having about 3 to about 18 (e.g., about 3, 4, 5, 6, 7, 8, 9, 10, 11, 12, 13, 14, 15, 16, 17, or 18) nucleotides that are complementary to the antisense region. The asymmetric hairpin siNA molecule can also comprise a 5′-terminal phosphate group that can be chemically modified. The loop portion of the asymmetric hairpin siNA molecule can comprise nucleotides, non-nucleotides, linker molecules, or conjugate molecules as described herein.

By “asymmetric duplex” as used herein is meant a siNA molecule having two separate strands comprising a sense region and an antisense region, wherein the sense region comprises fewer nucleotides than the antisense region to the extent that the sense region has enough complementary nucleotides to base pair with the antisense region and form a duplex. For example, an asymmetric duplex siNA molecule of the invention can comprise an antisense region having length sufficient to mediate RNAi in a T-cell (e.g., about 19 to about 22 (e.g., about 19, 20, 21, or 22) nucleotides) and a sense region having about 3 to about 18 (e.g., about 3, 4, 5, 6, 7, 8, 9, 10, 11, 12, 13, 14, 15, 16, 17, or 18) nucleotides that are complementary to the antisense region.

By “modulate gene expression” is meant that the expression of a target gene is upregulated or downregulated, which can include upregulation or downregulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA). For example, the term “modulate” can mean “inhibit,” but the use of the word “modulate” is not limited to this definition.

By “inhibit”, “down-regulate”, or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siNA) of the invention. In one embodiment, inhibition, down-regulation or reduction with an siNA molecule is below that level observed in the presence of an inactive or attenuated molecule. In another embodiment, inhibition, down-regulation, or reduction with siNA molecules is below that level observed in the presence of, for example, an siNA molecule with scrambled sequence or with mismatches. In another embodiment, inhibition, down-regulation, or reduction of gene expression with a nucleic acid molecule of the instant invention is greater in the presence of the nucleic acid molecule than in its absence.

Gene “silencing” refers to partial or complete loss-of-function through targeted inhibition of gene expression in a cell and may also be referred to as “knock down.” Depending on the circumstances and the biological problem to be addressed, it may be preferable to partially reduce gene expression. Alternatively, it might be desirable to reduce gene expression as much as possible. The extent of silencing may be determined by methods known in the art, some of which are summarized in International Publication No. WO 99/32619. Depending on the assay, quantification of gene expression permits detection of various amounts of inhibition that may be desired in certain embodiments of the invention, including prophylactic and therapeutic methods, which will be capable of knocking down target gene expression, in terms of mRNA levels or protein levels or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

The phrase “inhibiting expression of a target gene” refers to the ability of a siNA of the invention to initiate gene silencing of the target gene. To examine the extent of gene silencing, samples or assays of the organism of interest or cells in culture expressing a particular construct are compared to control samples lacking expression of the construct. Control samples (lacking construct expression) are assigned a relative value of 100%. Inhibition of expression of a target gene is achieved when the test value relative to the control is about 90%, often 50%, and in certain embodiments 25-0%. Suitable assays include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art.

By “subject” is meant an organism, tissue, or cell, which may include an organism as the subject or as a donor or recipient of explanted cells or the cells that are themselves subjects for siNA delivery. “Subject” therefore may refers to an organism, organ, tissue, or cell, including in vitro or ex vivo organ, tissue or cellular subjects, to which the nucleic acid molecules of the invention can be administered and enhanced by polynucleotide delivery-enhancing polypeptides described herein. Exemplary subjects include mammalian individuals or cells, for example human patients or cells.

As used herein “cell” is used in its usual biological sense, and does not refer to an entire multicellular organism, e.g., specifically does not refer to a human. The cell can be present in an organism, e.g., birds, plants and mammals such as humans, cows, sheep, apes, monkeys, swine, dogs, and cats. The cell can be prokaryotic (e.g., bacterial cell) or eukaryotic (e.g., mammalian or plant cell). The cell can be of somatic or germ line origin, totipotent or pluripotent, dividing or non-dividing. The cell can also be derived from or can comprise a gamete or embryo, a stem cell, or a fully differentiated cell.

By “vectors” is meant any nucleic acid- and/or viral-based technique used to deliver a desired nucleic acid.

By “RNA” is meant a molecule comprising at least one ribonucleotide residue. By “ribonucleotide” is meant a nucleotide with a hydroxyl group at the 2′ position of a .beta.-D-ribo-furanose moiety. The terms include double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siNA or internally, for example at one or more nucleotides of the RNA. Nucleotides in the RNA molecules of the instant invention can also comprise non-standard nucleotides, such as non-naturally occurring nucleotides or chemically synthesized nucleotides or deoxynucleotides. These altered RNAs can be referred to as analogs or analogs of naturally-occurring RNA.

By “highly conserved sequence region” is meant, a nucleotide sequence of one or more regions in a target gene does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “target nucleic acid” is meant any nucleic acid sequence whose expression or activity is to be modulated. The target nucleic acid can be DNA or RNA.

By “complementarity” is meant that a nucleic acid can form hydrogen bond(s) with another nucleic acid sequence by either traditional Watson-Crick or other non-traditional types. In reference to the nucleic molecules of the present invention, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner, et al., CSH Symp. Quant. Biol. LII, pp. 123-133, 1987; Frier, et al., Proc. Nat. Acad. Sci. USA 83:9373-9377, 1986; Turner, et al., J. Am. Chem. Soc. 109:3783-3785, 1987. A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonuelcotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, Nucleic Acids Research 29:2437-2447, 2001.

The term “acyclic nucleotide” as used herein refers to any nucleotide having an acyclic ribose sugar, for example where any of the ribose carbons (C1, C2, C3, C4, or C5), are independently or in combination absent from the nucleotide.

The term “biodegradable” as used herein, refers to degradation in a biological system, for example enzymatic degradation or chemical degradation.

The term “biologically active molecule” as used herein, refers to compounds or molecules that are capable of eliciting or modifying a biological response in a system. Non-limiting examples of biologically active siNA molecules either alone or in combination with other molecules contemplated by the instant invention include therapeutically active molecules such as antibodies, cholesterol, hormones, antivirals, peptides, proteins, chemotherapeutics, small molecules, vitamins, co-factors, nucleosides, nucleotides, oligonucleotides, enzymatic nucleic acids, antisense nucleic acids, triplex forming oligonucleotides, 2,5-A chimeras, siNA, dsRNA, allozymes, aptamers, decoys and analogs thereof. Biologically active molecules of the invention also include molecules capable of modulating the pharmacokinetics and/or pharmacodynamics of other biologically active molecules, for example, lipids and polymers such as polyamines, polyamides, polyethylene glycol and other polyethers.

The term “phospholipid” as used herein, refers to a hydrophobic molecule comprising at least one phosphorus group. For example, a phospholipid can comprise a phosphorus-containing group and saturated or unsaturated alkyl group, optionally substituted with OH, COOH, oxo, amine, or substituted or unsubstituted aryl groups.

By “cap structure” is meant chemical modifications, which have been incorporated at either terminus of the oligonucleotide (see, for example, Adamic, et al., U.S. Pat. No. 5,998,203, incorporated by reference herein). These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Lyer, Tetrahedron 49:1925, 1993, incorporated by reference herein).

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein, et al., International PCT Publication No. WO 92/07065; Usman, et al, International PCT Publication No. WO 93/15187; Uhlman & Peyman, supra, all are hereby incorporated by reference herein). There are several examples of modified nucleic acid bases known in the art as summarized by Limbach, et al, Nucleic Acids Res. 22:2183, 1994. Some of the non-limiting examples of base modifications that can be introduced into nucleic acid molecules include, inosine, purine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin, et al., Biochemistry 35:14090, 1996; Uhlman & Peyman, supra). By “modified bases” in this aspect is meant nucleotide bases other than adenine, guanine, cytosine and uracil at 1′ position or their equivalents.

By “target site” is meant a sequence within a target RNA that is “targeted” for cleavage mediated by a siNA construct which contains sequences within its antisense region that are complementary to the target sequence.

By “detectable level of cleavage” is meant cleavage of target RNA (and formation of cleaved product RNAs) to an extent sufficient to discern cleavage products above the background of RNAs produced by random degradation of the target RNA. Production of cleavage products from 1-5% of the target RNA is sufficient to detect above the background for most methods of detection.

By “biological system” is meant, material, in a purified or unpurified form, from biological sources, including but not limited to human, animal, plant, insect, bacterial, viral or other sources, wherein the system comprises the components required for RNAi acitivity. The term “biological system” includes, for example, a cell, tissue, or organism, or extract thereof. The term biological system also includes reconstituted RNAi systems that can be used in an in vitro setting.

The term “biodegradable linker” as used herein, refers to a nucleic acid or non-nucleic acid linker molecule that is designed as a biodegradable linker to connect one molecule to another molecule, for example, a biologically active molecule to a siNA molecule of the invention or the sense and antisense strands of a siNA molecule of the invention. The biodegradable linker is designed such that its stability can be modulated for a particular purpose, such as delivery to a particular tissue or cell type. The stability of a nucleic acid-based biodegradable linker molecule can be modulated by using various chemistries, for example combinations of ribonucleotides, deoxyribonucleotides, and chemically-modified nucleotides, such as 2′-O-methyl, 2′-fluoro, 2′-amino, 2′-O-amino, 2′-C-allyl, 2′-O-allyl, and other 2′-modified or base modified nucleotides. The biodegradable nucleic acid linker molecule can be a dimer, trimer, tetramer or longer nucleic acid molecule, for example, an oligonucleotide of about 2, 3, 4, 5, 6, 7, 8, 9, 10, 11, 12, 13, 14, 15, 16, 17, 18, 19, or nucleotides in length, or can comprise a single nucleotide with a phosphorus-based linkage, for example, a phosphoramidate or phosphodiester linkage. The biodegradable nucleic acid linker molecule can also comprise nucleic acid backbone, nucleic acid sugar, or nucleic acid base modifications.

By “abasic” is meant sugar moieties lacking a base or having other chemical groups in place of a base at the 1′ position, see for example Adamic, et al., U.S. Pat. No. 5,998,203.

By “unmodified nucleoside” is meant one of the bases adenine, cytosine, guanine, thymine, or uracil joined to the 1′ carbon of .beta.-D-ribo-furanose.

By “modified nucleoside” is meant any nucleotide base which contains a modification in the chemical structure of an unmodified nucleotide base, sugar and/or phosphate. Non-limiting examples of modified nucleotides are shown by Formulae I-VII and/or other modifications described herein.

In connection with 2′-modified nucleotides as described for the present invention, by “amino” is meant 2′—NH₂ or 2′-O—NH₂, which can be modified or unmodified. Such modified groups are described, for example, in Eckstein, et al., U.S. Pat. No. 5,672,695 and Matulic-Adamic, et al., U.S. Pat. No. 6,248,878.

The siNA molecules can be complexed with cationic lipids, packaged within liposomes, or otherwise delivered to target cells or tissues. The nucleic acid or nucleic acid complexes can be locally administered to through injection, infusion pump or stent, with or without their incorporation in biopolymers. In another embodiment, polyethylene glycol (PEG) can be covalently attached to siNA compounds of the present invention, to the polynucleotide delivery-enhancing polypeptide, or both. The attached PEG can be any molecular weight, preferably from about 2,000 to about 50,000 Daltons (Da).

The sense region can be connected to the antisense region via a linker molecule, such as a polynucleotide linker or a non-nucleotide linker.

“Inverted repeat” refers to a nucleic acid sequence comprising a sense and an antisense element positioned so that they are able to form a double stranded siRNA when the repeat is transcribed. The inverted repeat may optionally include a linker or a heterologous sequence such as a self-cleaving ribozyme between the two elements of the repeat. The elements of the inverted repeat have a length sufficient to form a double stranded RNA. Typically, each element of the inverted repeat is about 15 to about 100 nucleotides in length, preferably about 20-30 base nucleotides, preferably about 20-25 nucleotides in length, e.g., 20, 21, 22, 23, 24, 25, 26, 27, 28, 29, or 30 nucleotides in length.

“Nucleic acid” refers to deoxyribonucleotides or ribonucleotides and polymers thereof in single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Large double-stranded RNA” refers to any double-stranded RNA having a size greater than about 40 base pairs (bp) for example, larger than 100 bp or more particularly larger than 300 bp. The sequence of a large dsRNA may represent a segment of a mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

The double-stranded structure may be formed by self-complementary RNA strand such as occurs for a hairpin or a micro RNA or by annealing of two distinct complementary RNA strands.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

“One or more dsRNAs” refers to dsRNAs that differ from each other on the basis of sequence.

“Target gene or mRNA” refers to any gene or mRNA of interest. Indeed any of the genes previously identified by genetics or by sequencing may represent a target. Target genes or mRNA may include developmental genes and regulatory genes as well as metabolic or structural genes or genes encoding enzymes. The target gene may be expressed in those cells in which a phenotype is being investigated or in an organism in a manner that directly or indirectly impacts a phenotypic characteristic. The target gene may be endogenous or exogenous. Such cells include any cell in the body of an adult or embryonic animal or plant including gamete or any isolated cell such as occurs in an immortal cell line or primary cell culture.

In this specification and the appended claims, the singular forms of “a”, “an” and “the” include plural reference unless the context clearly dictates otherwise.

The polypeptide PN73 represents a partial amino acid sequence corresponding at least in part to a partial sequence of a histone protein, for example of one or more of the following histones: histone H1, histone H2A, histone H2B, histone H3 or histone H4, or one or more polypeptide fragments or derivatives thereof comprising at least a partial sequence of a histone protein, typically at least 5-10 or 10-20 contiguous residues of a native histone protein. In exemplary embodiments, the histone polynucleotide delivery-enhancing polypeptide comprises a fragment of histone H2B, as exemplified by the polynucleotide delivery-enhancing polypeptide designated PN73 described herein below. In yet additional detailed embodiments, the polynucleotide delivery-enhancing polypeptide may be pegylated to improve stability and/or efficacy, particularly in the context of in vivo administration. The amino acid sequence of PN73 is shown below and it has a molecular weight of 4229.1 Daltons:

(SEQ ID NO: 100)

KGSKKAVTKAQKKDGKKRKRSRKESYSVYVYKVLKQ

Within additional embodiments of the invention, the polynucleotide delivery-enhancing polypeptide is selected or rationally designed to comprise an amphipathic amino acid sequence. For example, useful polynucleotide delivery-enhancing polypeptides may be selected which comprise a plurality of non-polar or hydrophobic amino acid residues that form a hydrophobic sequence domain or motif, linked to a plurality of charged amino acid residues that form a charged sequence domain or motif, yielding an amphipathic peptide.

In other embodiments, the polynucleotide delivery-enhancing polypeptide is selected to comprise a protein transduction domain or motif, and a fusogenic peptide domain or motif. A protein transduction domain is a peptide sequence that is able to insert into and preferably transit through the membrane of cells. A fusogenic peptide is a peptide that is able destabilize a lipid membrane, for example a plasma membrane or membrane surrounding an endosome, which may be enhanced at low pH. Exemplary fusogenic domains or motifs are found in a broad diversity of viral fusion proteins and in other proteins, for example fibroblast growth factor 4 (FGF4).

To rationally design polynucleotide delivery-enhancing polypeptides of the invention, a protein transduction domain is employed as a motif that will facilitate entry of the nucleic acid into a cell through the plasma membrane. In certain embodiments, the transported nucleic acid will be encapsulated in an endosome. The interior of endosomes has a low pH resulting in the fusogenic peptide motif destabilizing the membrane of the endosome. The destabilization and breakdown of the endosome membrane allows for the release of the siNA into the cytoplasm where the siNA can associate with a RISC complex and be directed to its target mRNA.

Examples of protein transduction domains for optional incorporation into polynucleotide delivery-enhancing polypeptides of the invention include:

1. TAT protein transduction domain (PTD) (SEQ ID NO: 1) KRRQRRR;

2. Penetratin PTD (SEQ ID NO: 2) RQIKIWFQNRRMKWKK;

3. VP22 PTD (SEQ ID NO: 3) DAATATRGRSAASRPTERPRAPARSASRPRRPVD;

4. Kaposi FGF signal sequences (SEQ ID NO: 4) AAVALLPAVLLALLAP, and SEQ ID NO: 5) AAVLLPVLLPVLLAAP;

5. uman β3 integrin signal sequence (SEQ ID NO: 6) VTVLALGALAGVGVG;

6. gp41 fusion sequence (SEQ ID NO: 7) GALFLGWLGAAGSTMGA;

7. Caiman crocodylus Ig(v) light chain (SEQ ID NO: 8) MGLGLHLLVLAAALQGA;

8. hCT-derived peptide (SEQ ID NO: 9) LGTYTQDFNKFHTFPQTAIGVGAP;

9. Transportan (SEQ ID NO: 10) GWTLNSAGYLLKINLKALAALAKKIL;

10. Loligomer (SEQ ID NO: 11) TPPKKKRKVEDPKKKK;

11. Arginine peptide (SEQ ID NO: 12) RRRRRRR; and

12. Amphiphilic model peptide (SEQ ID NO: 13) KLALKLALKALKAALKLA.

Examples of viral fusion peptides fusogenic domains for optional incorporation into polynucleotide delivery-enhancing polypeptides of the invention include:

1. Influenza HA2 (SEQ ID NO: 14) GLFGAIAGFIENGWEG;

2. Sendai F1 (SEQ ID NO: 15) FFGAVIGTIALGVATA;

3. Respiratory Syncytial virus F1 (SEQ ID NO: 16) FLGFLLGVGSAIASGV;

4. HIV gp41 (SEQ ID NO: 17) GVFVLGFLGFLATAGS; and

5. Ebola GP2 (SEQ ID NO: 18) GAAIGLAWIPYFGPAA.

Within yet additional embodiments of the invention, polynucleotide delivery-enhancing polypeptides are provided that incorporate a DNA-binding domain or motif which facilitates polypeptide-siNA complex formation and/or enhances delivery of siNAs within the methods and compositions of the invention. Exemplary DNA binding domains in this context include various “zinc finger” domains as described for DNA-binding regulatory proteins and other proteins identified in Table 1, below (see, e.g., Simpson, et al., J. Biol. Chem. 278:28011-28018, 2003).

TABLE 1

Exemplary Zinc Finger Motifs of Different DNA-binding Proteins
C₂H₂ Zinc finger motif

| | | | | | |
|---|---|---|---|---|---|
| ....\|....\| | ....\|....\| | ....\|....\| | ....\|....\| | ....\|....\| | ....\|....\| |
| 665 | 675 | 685 | 695 | 705 | 715 |

| | | | | | | |
|---|---|---|---|---|---|---|
| Sp1 | ACTCPYCKDS | EGRGSG---- | DPGKKKDHIC | HIDGCGKVYG | KTSHLRAHLR | WHTGERFFMC |
| Sp2 | ACTCPNCKDG | EKRS------ | GEQGKKKHVC | HIPDCGKTFR | KTSLLRAHVR | LHTGERPFVC |
| Sp3 | ACTCPNCKEG | GGRGTN---- | -LGKKKQHIC | HIPGCGKVYG | KTSHLRAHLR | WHSGERPFVC |
| Sp4 | ACSCPNCREG | EGRGSN---- | EPGKKKQHIC | HIEGCGKVYG | KTSHLRAHLR | WHTGERPFIC |
| DrosBtd | RCTCPNCTNE | MSGLPPIVGP | DERGRKQHIC | HIPGCERLYG | KASHLKTHLR | WHTGERPFLC |
| DrosSp | TCDCPNCQEA | ERLGPAGV-- | HLRKKNIHSC | HIPGCGKVYG | KTSHLKAHLR | WHTGERPFVC |
| CeT22C8.5 | RCTCPNCKAI | KHG------- | DRGSQHTHLC | SVPGCGKTYK | KTSHLRAHLR | KHTGDRPFVC |
| Y40B1A.4 | PQISLKKKIF | FFIFSNFR-- | GDGKSRICIC | HL--CNKTYG | KTSHLRAHLR | GHAGNKPFAC |
| | [symbol] | | | | | |

Prosite pattern
C-x(2, 4)-C-x(12)-H-x(3)-H

*The table demonstrates a conservative zinc fingerer motif for double strand DNA binding which is characterized by the C-x(2,4)-C-x(12)-H-x(3)-H (SEQ ID NO. 97) motif pattern, which itself can be used to select and design additional polynucleotide delivery-enhancing polypeptides according to the invention.
**The sequences shown in Table 1, for Sp1, Sp2, Sp3, Sp4, DrosBtd, DrosSp, CeT22C8.5, and Y4pB1A.4, are herein assigned SEQ ID NOs: 19, 20, 21, 22, 23, 24, 25, and 26, respectively.

Alternative DNA binding domains useful for constructing polynucleotide delivery-enhancing polypeptides of the invention include, for example, portions of the HIV Tat protein sequence (see, Examples, below).

Within exemplary embodiments of the invention described herein below, polynucleotide delivery-enhancing polypeptides may be rationally designed and constructed by combining any of the foregoing structural elements, domains or motifs into a single polypeptide effective to mediate enhanced delivery of siNAs into target cells. For example, a protein transduction domain of the TAT polypeptide was fused to the N-terminal 20 amino acids of the influenza virus hemagglutinin protein, termed HA2, to yield one exemplary polynucleotide delivery-enhancing polypeptide herein. Various other polynucleotide delivery-enhancing polypeptide constructs are provided in the instant disclosure, evincing that the concepts of the invention are broadly applicable to create and use a diverse assemblage of effective polynucleotide delivery-enhancing polypeptides for enhancing siNA delivery.

Yet additional exemplary polynucleotide delivery-enhancing polypeptides within the invention may be selected from the following peptides:

(SEQ ID NO: 27)

WWETWKPFQCRICMRNFSTRQARRNHRRRHR;

(SEQ ID NO: 28)

GKINLKALAALAKKIL,

(SEQ ID NO: 29)

RVIRVWFQNKRCKDKK,

(SEQ ID NO: 30)

GRKKRRQRRRPPQGRKKRRQRRRPPQGRKKRRQRRRPPQ,

(SEQ ID NO: 31)

GEQIAQLIAGYIDIILKKKKSK,
Poly Lys-Trp, 4:1, MW 20,000-50,000;
and
Poly Orn-Trp, 4:1, MW 20,000-50,000.

Additional polynucleotide delivery-enhancing polypeptides that are useful within the compositions and methods herein comprise all or part of the mellitin protein sequence.

Charged Molecules

Examples of organic cations for use within the invention include, but are not limited to: ammonium hydroxide, D-arginine, L-arginine, t-butylamine, calcium acetate hydrate, calcium carbonate, calcium DL-malate, calcium hydroxide, choline, dethanolamine, ethylenediamine, glycine, L-histidine, L-lysine, magnesium hydroxide, N-methyl-D-glucamine, L-ornithine hydrochloride, potassium hydroxide, procaine hydrochloride, L-proline, pyridoxine, L-serine, sodium hydroxide, DL-triptophan, tromethamine, L-tyrosine, L-valine, camitine, taurine, creatine malate, arginine alpha keto glutarate, ornithine alpha keto glutarate, spermine acetate, and spermidine chloride.

Examples of organic anions for use within the invention include, but are not limited to: acetic acid, adamantoic acid, alpha keto glutaric acid, D-aspartic acid, L-aspartic acid, benzenesulfonic acid, benzoic acid, 10-camphorsulfunic acid, citric acid, 1,2-ethanedisulfonic acid, fumaric acid, D-gluconic acid, D-glucuronic acid, glucaric acid, D-glutamic acid, L-glutamic acid, glutaric acid, glycolic acid, hippuric acid, hydrobromic acid, hydrochloric acid, 1-hydroxyl-2-napthoic acid, lactobioinic acid, maleic acid, L-malic acid, mandelic acid, methanesulfonic aicd, mucic acid, 1,5 napthalenedisulfonic acid tetrahydrate, 2-napthalenesulfonic acid, nitric acid, oleic acid, pamoic acid, phosphoric acid, p-toluenesulfonic acid hydrate, D-saccharic acid monopotassium salt, salicylic acid, stearic acid, succinic acid, sulfuric acid, tannic acid, D-tartaric acid, L-tartaric acid, and other relate sugar carboxylate anions.

All publications, references, patents, patent publications and patent applications cited herein are each hereby specifically incorporated by reference in their entirety.

While this invention has been described in relation to certain embodiments, and many details have been set forth for purposes of illustration, it will be apparent to those skilled in the art that this invention includes additional embodiments, and that some of the details described herein may be varied considerably without departing from this invention. This invention includes such additional embodiments, modifications and equivalents. In particular, this invention includes any combination of the features, terms, or elements of the various illustrative components and examples.

The use herein of the terms “a,” “an,” “the,” and similar terms in describing the invention, and in the claims, are to be construed to include both the singular and the plural. The terms “comprising,” “having,” “including,” and “containing” are to be construed as open-ended terms which mean, for example, “including, but not limited to.” Recitation of a range of values herein refers individually to each separate value falling within the range as if it were individually recited herein, whether or not some of the values within the range are expressly recited. Specific values employed herein will be understood as exemplary and not to limit the scope of the invention.

Definitions of technical terms provided herein should be construed to include without recitation those meanings associated with these terms known to those skilled in the art, and are not intended to limit the scope of the invention.

The examples given herein, and the exemplary language used herein are solely for the purpose of illustration, and are not intended to limit the scope of the invention.

When a list of examples is given, such as a list of compounds or molecules suitable for this invention, it will be apparent to those skilled in the art that mixtures of the listed compounds or molecules are also suitable.

### EXAMPLES

### Example 1

### Low Concentrations of LC20 siRNA/PN73 Peptide Complex Precipitate Readily from Solution

The present example exemplifies the intrinsic instability of the LC20 siRNA/PN73 peptide complex at a concentration of 100 μM in a phosphate buffered saline (PBS) solution. The solution contains 250 μg/mL LC20 siRNA and 400 μg/mL PN73 peptide. Upon mixing LC20 siRNA and PN73 in PBS, this formulation immediately shows extensive turbidity and varied levels of precipitation with occlusive particulate contamination visible with the naked eye. In addition, characterization of the complex by static laser light scattering shows the presence of particular matter. As a result of the promiscuous aggregation of this complex, the LC20/PN73 complex is difficult to analyze by size exclusion chromatography. Lastly, a visible pellet is observed after centrifugation of the mixture, which is refractory to resuspension in water indicating the complex is highly insoluble. Analysis of the supernatant by UV spectrophotometry (UV260) shows a nearly 50-fold decrease in LC20 siRNA concentration in solution relative to the 250 μg/mL starting concentration.

The following examples explain various compositions and methods that stabilize the LC20 siRNA/PN73 peptide complex in solution, provide solutions of complexes which contain little or no aggregated particles of the complexes, and further provide methods to modify the complexes and increase their molecular size.

### Example 2

### The Addition of Various Organic Salt Competitors Creates LC20 siRNA/PN73 Peptide Complex Stability

In this example, the efficacy of various organic cationic and anionic competitors to create LC20 siRNA/PN73 peptide complex stability was tested. An intrinsic characteristic of the PN73 peptide is to aggregate and form large complexes. The addition of the LC20\siRNA reduces this aggregation; however, it does not prevent it nor reduce it significantly. Thus, an array of candidate organic cationic and anionic competitors were tested to determine if they could further reduce aggregation and promote LC20 siRNA/PN73 peptide complex stability in solution.

The ability of the organic salt competitor to promote complex stability was determined by the presence or absence of particle formation as measured by the naked eye. A visibly clear solution indicated that the salt competitor created LC20 siRNA/PN73 peptide complex stability. Further, all samples were analyzed by size exclusion chromatography coupled with an ultraviolet (UV) detector and a static laser light scattering detector (see Example 3). All experiments were performed in a final volume of 0.5 mL to 2.0 mL phosphate buffered saline at pH 7.2 with 17.5 μM LC20 siRNA and 95 μM PN73 (5:1 stoichiometry of PN73 peptide to LC20 siRNA). The working concentration of the LC20 siRNA/PN73 peptide complex was 100 μM.

This study examines whether the order of addition of the LC20 siRNA and the PN73 peptide to the organic salt competitor is a factor in maximizing LC20 siRNA/PN73 peptide complex stability. The following organic cations were used in this study: N-methyl-D-glucamine (NMDG), trimethylethanolamine (Choline), arginine, and spermine. They were chosen because they are well characterized and known to be safe for pharmaceutical salts. NMDG and arginine were tested with a glutamate anion while trimethylethanolamine was tested with a chloride anion. Spermine was tested with an acetate anion. Each salt was tested at a 100 mM, 10 mM and 1 mM concentration. This concentration range was chosen to promote stability for siRNA/PN73 and provide for an isotonic solution.

The PN73 peptide was mixed with 100 mM, 10 mM or 1 mM of the salt competitor followed by the addition of the LC20 siRNA. The contra experiment was performed whereby the LC20 siRNA was mixed first with the organic salt competitor followed then by the addition of the PN73 peptide. Both methods resulted in a clear solution indicating that the tested salt competitors can prevent LC20 siRNA/PN73 peptide complex aggregation and the order of addition of the organic salt competitor is not relevant to maximize complex stability in solution.

### Example 3

### Physical Characterization of the Organic Salt with the LC20 siRNA/PN73 Peptide Complex

In this example, size exclusion chromatography (SEC) coupled with an ultraviolet detector (UV 260 nm) and static laser light scattering (LS) detector was used to characterize the physical properties of the LC20 siRNA/PN73 peptide complex in the presence or absence of the organic salt. In addition, the phosphate/nitrogen (P/N) charge ratio for LC20 siRNA/PN73 was calculated.

Size Exclusion Chromatography/UV Detection/LS Detection

PN73 in monomeric form is 4 kiloDaltons (kDA); however an intrinsic property of this peptide is to aggregate and form large complexes in solution. An initial study was performed to analyze the physical properties of PN73, without LC20 siRNA, in the presence and absence of 100 mM NMDG-glutamate salt or 9% sorbitol (no salt environment). In the presence of 9% sorbitol, a UV trace with two overlapping peaks was observed at approximately 9 minutes. The LS signal showed that the molecular weight of the species that eluted from the size exclusion column was approximately 3 megaDaltons indicating that a significant amount of aggregation occurred after PN73 passed through the size exclusion column. In contrast, in the presence of 100 mM NMDG-glutamate salt, two distinct adjacent UV traces were observed indicating two distinct species of PN73 were present. The LS signal indicated that one species was approximately 3 megaDaltons, representing a large PN73 aggregate, and the other approximately 40 kDa. The 40 kDa molecular weight species indicates that the presence of 100 mM NMDG-glutamate salt reduces PN73 aggregation significantly. Next, the ability of NMDG-glutamate and other organic salts to reduced PN73 aggregation in the presence of LC20 siRNA was characterized by SEC-UV/LS.

LC20 siRNA/PN73 aggregation was characterized in the presence or absence of NMDG-glutamate by SEC-UV/LS. In the absence of NMDG-glutamate, a two overlapping UV traces were observed at 9 minutes which represented dissociated LC20 siRNA and PN73 molecules. In contrast, in the presence of 100 mM, 10 mM or 1 mM NMDG-glutamate, an additional UV trace was observed at approximately 5 minutes, indicating a stable LC20 siRNA/PN73 complex was present. The LS trace showed that a larger molecular weight species was created with LC20 siRNA and PN73 in the presence of NMDG-glutamate than in the absence of NMDG-glutamate. These data indicate that NMDG-glutamate is an effective stabilizer of the LC20 siRNA/PN73 complex in solution at concentrations of 100 mM, 10 mM, and 1 mM.

A similar SEC-UV/LS profile was observed with 100 mM, 10 mM, and 1 mM arginine glutamate indicating that, like NMDG-glutamate, arginine glutamate is an effective stabilizer at these salt concentrations. However, the LS trace for 150 mM arginine glutamate showed a significant presence of intermediary aggregating molecules between the 9 minute and 5 minute UV traces. Thus, arginine glutamate is not an effective stabilizer at a 150 mM concentration.

Spermine acetate at 10 mM and 1 mM showed a similar SEC-UV/LS profile indicating it too is an effective LC20 siRNA/PN73 stabilizer at 10 mM and 1 mM. In contrast, LC20 siRNA and PN73 in the presence of 100 mM spermine acetate showed an additional UV trace at approximately 7 minutes and a significantly reduced UV trace at 5 minutes (i.e., the peak corresponding to a stable LC20 siRNA/PN73 complex). This data indicates that 100 mM spermine acetate dissociates the LC20 siRNA/PN73 peptide complex. Thus, spermine acetate is an effective stabilizer of the LC20 siRNA/PN73 complex at a concentration of more than 1 mM but less than 100 mM.

Choline chloride showed UV traces similar to the other organic salts tested; however, the LS trace for choline chloride at 100 mM, 10 mM and 1 mM showed a significant presence of intermediary aggregating molecules between the 9 minute and 5 minute UV traces. Therefore, choline chloride can stabilize the LC20 siRNA/PN73 peptide complex, but it also allows for the formation of unwanted aggregates in solution. One interpretation of this is that choline chloride prevents LC20 siRNA/PN73 peptide complex aggregation in a time dependent manner. Nonetheless, it may not be suitable as a stabilizer at the concentrations tested.

Charge Ratio Calculations for LC20/PN73

The phosphate (P) to nitrogen (N) charge ratio (P/N) was calculated for the LC20/PN73 complex. The molar concentration of phosphate anions in LC20 siRNA was calculated to be 720 μM or 0.72 mM (P) and the molar concentration of the protonated nitrogen cations in PN73 was calculated to be 1.23 mM. At a 1:1 stoichiometry, all LC20 siRNA/PN73 peptide conjugates have a P/N ratio of 3 indicating that the complex forms large aggregates over time making it ineffective as delivery agent. However, as presented in the above Examples, the addition of cationic and anionic salts with LC20 siRNA/PN73 prevents aggregations and promotes complex stability in solution.

### Example 4

### Thermal Method for Modifying the siRNA/Peptide Complex

The present example demonstrates that thermal treatment of the siRNA/peptide complex modifies the complex as shown by gel electrophoresis. This method increases the temperature of the siRNA/peptide complex from approximately room temperature to 55° C. in order to enable annealing of the peptide in a condensed manner with the siRNA. One variation (variation A) of this thermal method included heating the siRNA/peptide complex up to about 55° C. at approximately 1° C./minute and maintaining that temperature for 10 to 30 minutes. The temperature was then decreased to about room temperature at approximately 1° C./minute. A second variation (variation B) of the thermal method included placing the siRNA/peptide complex sample into an environment (e.g., heating block or water bath) at or about 55° C. for 10 to 30 minutes and then decreasing the temperature of the environment to about room temperature at approximately 1° C./minute. For the purposes of the instant example, a non-thermal treated siRNA/peptide complex was used as a control.

The ratio by weight of the siRNA to peptide for the instant example was 62.5 μg/ml to 100 μg/ml. The materials and reagents used in the instant example are shown below in Table 2.

TABLE 2

| Reagent | Manufacturer | Lot # |
|---|---|---|
| siRNA: LC20Md8 | Qiagen ™ | DX0110 |
| | | B324P69 |
| Peptide: PN602 (Peptide) | | |
| DEPC-Water | | |
| Nuclease-Free Water | Ambion ™ | 065P053618A |
| TBE-Urea 15% pre-cast Gel | BioRad ™ | L020206AC |
| 2× Sample Buffer (Denaturing) | Ambion ™ | n/a |

PN602 is an acetylated form of the peptide named PN73.

The nucleotide sequence and nucleotide modifications of the LC20Md8 siRNA molecules are as follows:

(SEQ ID NO: 98)

5′-G^{MeO}G^{MeO}GT^{r}CGGAACCCAAGCT^{r}T^{r}A dTdT -3′

(SEQ ID NO: 99)

3′- dAdT CCCAGCCT^{r}T^{r}GGGT^{r}T^{r}CGAA^{MeO}U^{MeO}-p -5′

whereby, a 2′-O-methyl modified ribonucleotide is indicated by a “MeO” above the ribonucleotide (e.g., N^{MeO} where N is the ribonucleotide). A ribothymidine is indicated by an “r” above the ribonucleotide (e.g., N^{r}).

Polyacrylamide gel electrophoresis (denaturing conditions) and ethidium bromide staining were used to characterize the effect of thermal treatment on the siRNA/peptide complex. A 20 μl sample of siRNA alone (62.5 μg/ml), the peptide alone (100 μg/ml), the non-thermal treated siRNA/peptide complex, the pre-thermal treated siRNA/peptide complex by variation A, the post-thermal treated siRNA/peptide complex by variation A, the pre-thermal treated siRNA/peptide complex by variation B and the post-thermal treated siRNA/peptide complex by variation B were assayed on a TBE-Urea 15% polyacrylamide gel. The pre-thermal treated siRNA/peptide complex samples for both variations A and B served as controls to determine whether subjecting the complex to a heating and cooling cycle modified the complex as measured by gel electrophoresis. The pre-thermal samples were created at the same time as the post-thermal samples but never subjected to the heating and cooling cycle. These control samples were incubated at room temperature for the same length of time the post-thermal samples were subjected to the heating and cooling cycles.

The migration patterns of the samples on the polyacrylamide gel were visualized by exposing the ethidium bromide stained gel to UV light. The migration pattern of the siRNA/peptide complex on a 15% TBE-Urea polyacrlyamide gel after thermal treatment (“heating and cooling”) of the complex was obtained. As expected, the siRNA alone (lane 2) migrated on the gel as a single distinct band while the peptide alone (lane 3) did not generate a band. The non-treated siRNA/peptide complex (lane 4) migrated as two distinct bands indicating two different molecular weight species were present. The migration pattern of the lower molecular weight band matched that of the siRNA alone sample, indicating that the lower molecular weight band was likely free siRNA. The presence of the higher molecular weight band indicates that the migration of the siRNA molecule was retarded, likely due to the presence of the peptide (siRNA/peptide complex).

The pre-thermal treated samples for variation A and variation B (lanes 7 and 8, respectively) and the post-thermal treated samples for variation A and variation B (lanes 5 and 6, respectively) showed that the siRNA/peptide complexes also migrated as two distinct bands. However, a change in intensity of the higher molecular weight bands of the post-thermal treated variations A and B compared to the pre-thermal treated variations A and B siRNA/peptide complex samples was observed.

These data indicated that the thermal method of treatment (“heating and cooling method”) modified the siRNA/peptide complex as evidenced by the broader and more intense size higher molecular weight band on the polyacrylamide gel. These data further show that incubation of the siRNA/peptide complex at room temperature (pre-thermal treated control samples) did not result in the same broad and intense higher molecular weight band, confirming that thermal treatment is responsible for the modified siRNA/polypeptide complex observed on the polyacrylamide gel.

### Example 5

### Dialysis Method for Modifying the siRNA/Peptide Complex

The present example demonstrates that the removal of high concentrations of various salt forms of the siRNA/peptide complex via dialysis to isotonic conditions modifies the complex as shown by gel electrophoresis. The monovalent salt sodium chloride and the divalent cationic chloride salts of calcium, zinc and magnesium were used in the instant example. Urea was also used in the instant example. The different salt forms of the siRNA/peptide complex were prepared by making the complex at high salt concentrations with the respective salt with the purpose of dissociating the ionically bound complex and then slowly removing that salt through dialysis. The goal of the process is to generate “optimized” or highly stable siRNA/peptide complexes. The method used to perform the dialysis for each salt is described.

The siRNA to polypeptide ratio was 1:5 molar (1.6 charge) or 62.5 μg/ml to 100 μg/ml by weight. The siRNA molecule (LC20Md8) and peptide (PN602) shown in Example 4 is used to form the complex of the instant example. The same ratio and siRNA and polypeptide were used for each of the following methods detailed below in the instant example unless specified otherwise.

Dialysis from Sodium Chloride (NaCl)

Dialyzing a high concentration of sodium chloride to allow siRNA/peptide complexes to relax into an optimized structure once normal saline conditions was achieved. A 3.5 KDa MWCO membrane (Pierce Slide-A-Lyzer) was used to perform the dialysis. One milliter of siRNA/peptide complex was incubated alone for 30 minutes. Following this incubation, 4M NaCl was added to the complex to achieve a final concentration of 1.5 M NaCl and then 2×400 μL was added to two separate dialysis cassettes and dialyzed against either 1× phosphate buffered saline (PBS) or 0.1×PBS (without Ca²⁺ or Mg²⁺). After 1.5 hours of dialysis, a small sample of the dialysis product was set aside for analysis by gel electrophoresis. The dialysis buffer was exchanged and the samples were dialyzed for an additional 4.5 hours.

Polyacrylamide gel electrophoresis and ethidium bromide staining were used to characterize the effect of dialysis on the siRNA/peptide complex. A 10 μL aliquot of the siRNA alone, the siRNA/peptide complex in 1.5M NaCl, the siRNA/polypeptide complex after 1.5 hours of dialysis with 0.1×PBS, the siRNA/polypeptide complex after 1.5 hours of dialysis with 1×PBS, the siRNA/peptide complex after 4.5 hours of dialysis with 0.1×PBS and the siRNA/peptide complex after 4.5 hours of dialysis with 1×PBS were analyzed analyzed by gel electrophoresis on both a urea denaturing gel (15% TBE-Urea) and a native gel (15% PAGE-TBE). The migration patterns of the samples on the polyacrylamide gels were visualized by exposing the ethidium bromide stained gels to UV light.

The migration pattern of the siRNA/peptide complex on a 15% TBE-Urea polyacrylamide gel after dialysis against sodium chloride was obtained. As expected, the siRNA alone (lane 1) migrated on the urea denaturing gel as a single distinct band. The non-dialyzed siRNA/peptide complex in 1.5 M NaCl (lane 2) migrated as two distinct bands on the urea denaturing gel indicating two different molecular weight species were present. The migration pattern of the lower molecular weight band matched that of the siRNA alone, indicating that the lower molecular weight band was likely free siRNA. The presence of the higher molecular weight band indicated that the migration of the siRNA molecule was retarded, likely due to the presence of the peptide (siRNA/peptide complex).

The migration pattern for non-dialyzed siRNA/peptide complex in 1.5 M NaCl showed that the complex resolves itself as if it were in the “normal” complex, suggesting that during electrophoresis in high sodium chloride the rapid migration of the small ion of sodium and chloride results in the rapid reformation of a complex. Both siRNA/peptide complexes which were subjected to 1.5 hours of dialysis with 1×PBS (lane 4) or 0.1×PBS (lane 3) migrated as two distinct bands similar to the non-dialyzed siRNA/peptide complex. However, the bands resulting from the 1.5 hour dialyzed samples showed lower intensity than the non-dialyzed siRNA/peptide complex sample. This result was likely due to a leaky dialysis cassette or an osmotic influx of extra water. The siRNA/peptide complex which was subjected to 4.5 hours of dialysis with 1×PBS (lane 5) also migrated as two distinct bands on the urea denaturing gel, but the higher molecular weight band migrated differently from that of the higher molecular weight band of the non-dialyzed siRNA/peptide complex. Lane 6 did not contain a band, likely due to a leaky dialysis cassette. These data indicate that prolonged dialysis (4.5 hours) in 1.5 NaCl against 1×PBS creates a different species of the siRNA/peptide complex compared to that of the species observed with the non-dialyzed siRNA/peptide complex.

These data indicate that prolonged dialysis (4.5 hours) of the siRNA/peptide complex from 1.5M NaCl modifies the siRNA/peptide complex as evidenced by the altered migration pattern of the siRNA on a urea denaturing gel.

Dialysis from Calcium Chloride (CaCl₂)

The divalent salt calcium chloride was used in dialysis to modify the siRNA/peptide complex. Dialysis was performed against 14 mM and 70 mM CaCl₂.

The materials and reagents used are shown below in Table 3.

TABLE 3

| Reagent | Grade | Manufacturer | Lot # |
|---|---|---|---|
| CaCl₂ | Research | Sigma ™ | 39H0085 |
| Snake Skin, 3.5 kDa | Research | Pierce Biotech ™ | FC69146 |
| MWCO | | | |
| DEPC Water | Research | | |
| Nuclease-Free Water | Research | Ambion ™ | 065P053618A |
| TBE-Urea 15% pre-cast | Research | BioRad ™ | L020206AC |
| Gel | | | |
| 2× Sample Buffer | Research | Ambion ™ | n/a |
| (Denaturing) | | | |

The siRNA and peptide were allowed to complex for 30 minutes at room temperature and then 0.5 volume samples were used to dialyze in a 3.5 kDa MWCo dialysis tube against 14 mM or 70 mM CaCl₂ buffered with PBS. After two hours of dialysis, samples were taken, mixed with sample buffer and then analyzed by gel electrophoresis.

Polyacrylamide gel electrophoresis and ethidium bromide staining were used to characterize the effect of dialysis on the siRNA/peptide complex. A sample of the siRNA alone, the untreated siRNA/peptide complex at a 1:5 ratio (lane 2), the untreated siRNA/peptide complex at a 1:10 ratio (lane 3), the untreated siRNA/peptide complex at a 1:20 ratio (lane 4), the siRNA/peptide complex at a 1:5 with 50% mouse plasma (lane 5), the siRNA/peptide complex at a 1:10 ratio with 50% mouse plasma (lane 6), the siRNA/peptide complex at a 1:20 ratio with 50% mouse plasma (lane 7), the siRNA/peptide complex at a 1:5 ratio in 1.5M NaCl before dialysis with CaCl₂ (lane 9), the siRNA/peptide complex at a 1:5 ratio after dialysis with 14 mM CaCl₂ (lane 11) and the siRNA/peptide complex at a 1:5 ratio after dialysis with 70 mM CaCl₂ (lane 12) were analyzed by gel electrophoresis on a urea denaturing gel (15% TBE-Urea). The migration patterns of the samples on the polyacrylamide gels were visualized by exposing the ethidium bromide stained gels to UV light.

The migration pattern of the siRNA/peptide complexes on a 15% TBE-Urea polyacrylamide gel after dialysis against calcium chloride was obtained. As expected, the siRNA alone (lane 1) migrated on the urea denaturing gel as a distinct band (a smaller molecular weight band likely represented a degradation production of the siRNA). Lanes 2 through 4 showed two distinct bands on the urea denaturing gel indicating two different molecular weight species were present. The migration pattern of the lower molecular weight band matched that of the siRNA alone sample indicating that the lower molecular weight band was likely siRNA. The presence of the higher molecular weight band indicated that the migration of the siRNA molecule was retarded, likely due to the presence of the peptide (siRNA/peptide complex). Lanes 5 through 7 also showed three distinct bands indicating three different molecular weight species were present.

Lane 9 representing the siRNA/peptide complex at a 1:5 ratio in 1.5M NaCl before dialysis with CaCl₂ showed similar bands with similar migration pattern to the untreated siRNA/peptide complex at the varying ratios. Lanes 12 and 13 show the effect on the migration pattern of the solution containing the siRNA/peptide complex subjected to dialysis with calcium chloride. Lane 11 representing dialysis with 14 mM calcium chloride showed a single high molecular weight band while lane 12 representing dialysis with 70 mM calcium chloride showed three distinct molecular weight bands. The lower molecular weight band coincided with the band found in the intense band for siRNA alone (lane 1), while the high molecular weight band in lane 12 was similar in size to the mouse plasma treated siRNA/peptide complex (lanes 5, 6 and 7), which may be due to the presence of 2.5 mM calcium ion in the blood (mouse plasma) and additional components that may modify the siRNA/peptide complex and consequently alter its migration pattern.

These data indicated that dialysis of the siRNA/peptide complex with 70 mM calcium chloride modified the siRNA/peptide complex as evidenced by the altered migration pattern of the siRNA on a urea denaturing gel.

Dialysis from Zinc Chloride (ZnCl₂) and Magnesium Chloride (MgCl₂)

The divalent salts zinc chloride and magnesium chloride were used in dialysis to modify the siRNA/peptide complex. The dialysis method used herein for ZnCl₂ and MgCl₂ are similar to what was described above for NaCl and CaCl.

The materials and reagents used are shown below in Table 4.

TABLE 4

| Reagent | Grade | Manufacturer | Lot # |
|---|---|---|---|
| MgCl₂ | Research | Sigma ™ | UB0196 |
| ZnCl₂ | Research | Sigma ™ | SG1368 |
| 2.0 kDa MWCO Slide- | Research | Pierce Biotech ™ | G199825 |
| A-Lyzer cassettes | | | |
| DEPC-Water | Research | Nastech ™ | n/a |
| Nuclease-Free Water | Research | Ambion ™ | 065P053618A |
| TBE-Urea 15% pre-cast | Research | BioRad ™ | L020206AC |
| Gel | | | |
| 2× Sample Buffer | Research | Ambion | n/a |
| (Denaturing) | | | |

A 500 μL sample containing the siRNA and peptide at a ratio of 62.5 μg/mL to 100 μg/mL siRNA to peptide in 1.5 M NaCl (buffered with 10 mM phosphate, pH 7.2 (1:5 molar, 1.0 charge; final concentration corresponds to that of 0.25× of final dosing). Complex placed into sealed dialysis bag (Pierce Snake Skin®; 3.5 kDa MWCO), starting sample taken. The dialysis bag was placed into either 14 mM or 70 mM zinc chloride or 14 mM or 70 mM magnesium chloride dialysis solutions, incubated for 4 hours at room temp. Samples were removed and 2× sample buffer added, incubated at 65° C. and analyzed by gel electrophoresis on 15% Urea-TBE gel.

Polyacrylamide gel electrophoresis and ethidium bromide staining were used to characterize the effect of dialysis on the siRNA/peptide complex. A sample of the pre-dialysis siRNA/peptide complex (lane 1), the peptide alone (100 μg/mL; lane 2), the siRNA/peptide complex dialyzed with 14 mM MgCl₂ (lane 3), the siRNA/peptide complex dialyzed with 70 mM MgCl₂ (lane 4), the siRNA alone (62.5 μg/mL; lane 5), the siRNA/peptide complex dialyzed with 14 mM ZnCl₂ (lane 6) and the siRNA/peptide complex dialyzed with 70 mM ZnCl₂ (lane 7) were analyzed by gel electrophoresis on a urea denaturing gel (15% TBE-Urea). The migration patterns of the samples on the polyacrylamide gels were visualized by exposing the ethidium bromide stained gels to UV light.

The migration pattern of the siRNA/peptide complexes on a 15% TBE-Urea polyacrylamide gel after dialysis against zinc chloride alone or magnesium chloride alone was obtained. As expected, the siRNA alone (lane 5) migrated on the urea denaturing gel as a distinct band while the peptide alone (lane 2) did not generate a band. The pre-dialzyed siRNA/peptide complex sample showed two distinct molecular weight bands indicating two different molecular weight species were present. The migration pattern of the lower molecular weight band matched that of the siRNA alone (lane 5) indicating that the lower molecular weight band was likely free siRNA. The presence of the higher molecular weight band indicated that the migration of the siRNA molecule was retarded, likely due to the presence of the complex. The samples with siRNA/peptide complexes dialyzed against the 14 mM concentration of either salt showed a migration pattern similar to that of the non-diazlyed siRNA/peptide complex (lane 1). However, the samples dialyzed against the 70 mM concentration of either magnesium chloride (lane 4) or zinc chloride (lane 7) showed an additional band with a molecular weight greater than the free siRNA (lane 5).

These data indicated that dialysis of the siRNA/peptide complex with 70 mM zinc or magnesium chloride modified the siRNA/peptide complex as evidenced by the altered migration pattern of the siRNA on a urea denaturing gel.

Dialysis from Urea (Urea Shift)

Urea was used in dialysis to modify the siRNA/peptide complex. The materials and reagents used are shown below in Table 5.

TABLE 5

| Reagent | Grade | Manufacturer | Lot # |
|---|---|---|---|
| MgCl₂ | Research | Sigma ™ | UB0196 |
| ZnCl₂ | Research | Sigma ™ | SG1368 |
| 2.0 kDa MWCO Slide- | Research | Pierce Biotech ™ | G199825 |
| A-Lyzer cassettes | | | |
| DEPC Water | Research | | |
| Nuclease-Free Water | Research | Ambion ™ | 065P053618A |
| TBE-Urea 15% pre-cast | Research | BioRad ™ | L020206AC |
| Gel | | | |
| 2× Sample Buffer | Research | Ambion ™ | n/a |
| (Denaturing) | | | |

siRNA/peptide complexes were formed in a 500 μL volume with a 200 μg/mL to 400 μg/mL siRNA to peptide ratio (1:5 molar, 1.0 charge; final concentration corresponds to that of 0.25× of final dosing). The initial stock solution containing the siRNA/peptide complexes were subdivided into four portions of 125 μL each (then diluted 4-fold to 62.5/100 μg/mL at still a 1:5 molar ratio). Urea was used at the following molarities:

A—no urea control; B—2.5 M urea; C—5.0 M urea and D—7.5 M urea (samples taken of starting material). The solutions were then placed into separate dialysis slides and dialyzed, (12 hours) into a 1× phosphate buffered saline (PBS) or 1 M urea solution (samples were taken after 1 M urea dialysis). Solution dialysis cassettes were placed into 1×PBS for the final dialysis (6 hours), then final set of samples taken. To all samples, 0.5 volume of 2× sample buffer was added and incubated at 65° C. and then analyzed by gel electrophoresis on a 15% TBE-Urea gel.

Polyacrylamide gel electrophoresis and ethidium bromide staining were used to characterize the effect of dialysis on the siRNA/peptide complex. Samples of each treatment were analyzed by gel electrophoresis on a urea denaturing gel (15% TBE-Urea). The migration patterns of the samples on the polyacrylamide gels were visualized by exposing the ethidium bromide stained gels to UV light.

The migration pattern of the siRNA/peptide complexes on a 15% TBE-Urea polyacrylamide gel after dialysis against urea was obtained. The presence of urea with the siRNA/peptide complex sample (lane 5) generated a higher molecular weight band on the gel indicating that the presence of urea (7.5 M urea) drove the formation of a larger complex. Following dialysis with urea, the migration pattern of the siRNA/peptide complex samples indicated that the different urea starting concentrations did not have an effect on the siRNA/peptide complex.

These data indicated that dialysis of the siRNA/peptide complex to IM urea or 1×PBS did not modify the complex.

### Example 6

### Freeze-Thaw Method for Modifying the siRNA/Peptide Complex

The present example demonstrates that subjecting the siRNA/peptide complex to multiple freeze-thaw cycles modifies the physical properties of the complex as shown by gel electrophoresis. This method subjects the siRNA/peptide complex to one, two or four rounds of freeze/thaw (F/T) cycles. The F/T cycles include subjecting the samples to or about −80° C. and then increasing the temperature to or about room temperature (approximately 23° C.). The samples are maintained at the target temperature for approximately 30 minutes.

The ratio by weight of the siRNA to peptide for the instant example was 62.5 μg/ml to 100 μg/ml. The siRNA molecule (LC20Md8) and peptide (PN602) shown in Example 4 is used to form the complex of the instant example. The siRNA/polypeptide complex was made in a 100 μl final volume in either phosphate buffered saline (PBS), pH 7.2 or 0.1×PBS, pH 7.2. Twenty microliter aliquots were made from the 100 μl samples and were the subject of the F/T method described. A 20 μl not subject to the F/T method served as a control.

The materials and reagents used are shown below in Table 6.

TABLE 6

| Reagent | Grade | Manufacturer | Lot # |
|---|---|---|---|
| Urea | USP | Mallinckrodt ™ | 8642-Y29600 |
| Slide-A-Lyzer, 2 kDa | Research | Pierce Biotech ™ | GI99825 |
| MWCO | | | |
| DEPC-Water | Research | Nastech ™ | n/a |
| Nuclease-Free Water | Research | Ambion ™ | 065P053618A |
| TBE-Urea 15% pre-cast | Research | BioRad ™ | L020206AC |
| Gel | | | |
| 2× Sample Buffer | Research | Ambion ™ | n/a |
| (Denaturing) | | | |

Polyacrylamide gel electrophoresis (15% TBE Urea PAGE) and ethidium bromide staining were used to characterize the effect of the F/T treatment on the siRNA/peptide complex. A sample of the siRNA alone (lane 1), the siRNA/peptide complex in 1×PBS without a F/T treatment (lane 2), the siRNA/peptide complex in 1×PBS with one F/T treatment (lane 3), the siRNA/peptide complex in 1×PBS with two F/T treatments (lane 4), the siRNA/peptide complex in 1×PBS with four F/T treatments (lane 5), lane 6 was not loaded and was a blank, the siRNA/peptide complex in 0.1×PBS without a F/T treatment (lane 7), the siRNA/peptide complex in 0.1×PBS with one F/T treatment (lane 8), the siRNA/peptide complex in 0.1×PBS with two F/T treatments (lane 9) and the siRNA/peptide complex in 0.1×PBS with four F/T treatments (lane 10) were analyzed by gel electrophoresis on a urea denaturing gel (15% TBE-Urea). The migration patterns of the samples on the polyacrylamide gels were visualized by exposing the ethidium bromide stained gels to UV light.

The migration pattern of the siRNA/peptide complexes on a 15% TBE-Urea polyacrlyamide gel after a single or plurality of freeze-thaw treatments was obtained. As expected, the siRNA alone (lane 1) migrated on the gel as a distinct band. Lanes 2 through 5 and lanes 7 through 10 showed multiple bands indicating the presence of multiple molecular weight species. The migration pattern of the lower molecular weight band matched that of the siRNA alone, indicating that the lower molecular weight band was likely siRNA. The presence of the higher molecular weight bands indicated that the migration of the siRNA molecule was retarded, likely due to the presence of a peptide (siRNA/peptide complex). In contrast to the siRNA/peptide complex control samples, 1×PBS and 0.1×PBS not subjected to a F/T treatment, lanes 2 and 7, respectively, the siRNA/peptide complexes subjected to either one, two or four F/T treatment(s) showed a modified migration pattern. Specifically, the F/T treated siRNA/peptide complexes (lanes 3, 4, 5, 8, 9 and 10) showed additional high molecular weight bands not found in the control samples (lanes 2 and 7), indicating that all F/T treatments modified the siRNA/complex.

These data showed that subjecting the siRNA/peptide complex to a single or plurality of F/T treatments modified the complex as evidenced by the altered migration pattern on a polyacrylamide gel.

### Example 7

### pH Shift Method for Modifying the siRNA/Peptide Complex

The present example demonstrates that shifting the pH of a solution containing siRNA/peptide complexes modifies the physical properties of the complex as shown by gel electrophoresis. This method subjects the solution containing siRNA/peptide complexes to a pH shift. The pH shift is accomplished by placing the solution containing siRNA/peptide complexes into a dialysis bag and then incubating that bag for 30 minutes in PBS, pH 3.0 dialysis solution at room temperature. After the 30 minute incubation, a sample is taken for analysis by gel electrophoresis. The pH of the dialysis solution is then increased by one pH unit and the dialysis bag containing the solution with siRNA/peptide complexes is incubated again for 30 minutes at room temperature. Again, another sample is taken after this 30 minute incubation. The incremental pH increase of the dialysis solution with the 30 minutes incubation step and sample removal steps are repeated until the dialysis solution reaches pH 7.2 (the last pH increase is from pH 6.0 to pH 7.2). The collected samples are diluted in a half volume of 2× sample buffer and incubated at 65° C. and analyzed by gel electrophoresis.

The ratio by weight of the siRNA to peptide for the instant example was 62.5 μg/ml to 100 μg/ml. The siRNA molecule (LC20Md8) and peptide (PN602) shown in Example 4 is used to form the complex of the instant example.

The materials and reagents used are shown below in Table 7.

TABLE 7

| Reagent | Grade | Manufacturer | Lot # |
|---|---|---|---|
| CaCl₂ | Research | Sigma ™ | 39H0085 |
| Snake Skin, 3.5 kDa | Research | Pierce Biotech ™ | FC69146 |
| MWCO | | | |
| DEPC-Water | Research | Nastech ™ | n/a |
| Nuclease-Free Water | Research | Ambion ™ | 065P053618A |
| TBE-Urea 15% pre-cast | Research | BioRad ™ | L020206AC |
| Gel | | | |
| 2× Sample Buffer | Research | Ambion ™ | n/a |
| (Denaturing) | | | |

Polyacrylamide gel electrophoresis (15% TBE Urea PAGE) and ethidium bromide staining were used to characterize the effect of the pH shift treatment on the siRNA/peptide complex. A sample of the siRNA alone (lane 2), the non-treated siRNA/peptide complex (lane 1), lane 3 was not loaded and was blank, the siRNA/peptide at pH 3.0 time zero (lane 4), the siRNA/peptide complex at pH 3.0 after 30 minutes incubation (lane 5), the siRNA/peptide complex at pH 4.0 after 30 minutes incubation (lane 6), the siRNA/peptide at pH 5.0 after 30 minutes incubation (lane 7), the siRNA/peptide complex at pH 6.0 after 30 minutes incubation (lane 8) and the siRNA/peptide complex at pH 7.2 after 30 minutes incubation (lane 9) were analyzed by gel electrophoresis on a urea denaturing gel (15% TBE-Urea). The migration patterns of the samples on the polyacrylamide gels were visualized by exposing the ethidium bromide stained gels to UV light.

The migration pattern of the siRNA/peptide complexes on a 15% TBE-Urea polyacrlyamide gel after a pH shift of the complex was obtained. As expected, the siRNA alone (lane 2) migrated as a distinct band. The non-treated siRNA/peptide complex (lane 1) migrated as two distinct bands, indicating two different molecular weight species were present. The migration pattern of the lower molecular weight band matched that of the siRNA alone, indicating that the lower molecular weight band was likely free siRNA. The presence of the higher molecular weight band indicated that the migration of the siRNA molecule was retarded, likely due to the presence of the peptide (siRNA/peptide complex). The samples of siRNA/peptide complex with lower relative pH (lanes 4 through 5) showed a banding pattern on the gel distinct from that of the non-treated siRNA/peptide complex sample. Additionally, this distinct banding pattern disappeared and the migration of the siRNA/peptide complex samples resembled that of the non-treated samples as the pH of the samples approached neutral (pH 7.2; lane 7).

These data indicated that the siRNA/peptide complex was modified in the lower pH ranges (from about 3 to about 7.0) as evidenced by a distinct banding pattern on a polyacrylamide gel.

### Example 8

### Hold Time Method for Modifying the siRNA/Peptide Complex

The present example demonstrates that subjecting the siRNA/peptide complex to prolonged, for example six hours, ambient room temperatures does not modify the complex as evidence by gel electrophoresis. This method addressed the impact on the relaxation kinetics of the siRNA/peptide complex without addition of an external agent or energy source, as exemplified in the prior example sections. The “hold time” method determined whether energetics associated with relaxation of the complex requires external drivers to facilitate or expedite the transition of that complex. Other treatments of the siRNA/peptide complex were analyzed by gel electrophoresis in parallel as comparators.

The ratio by weight of the siRNA to peptide for the instant example was 62.5 μg/ml to 100 μg/ml. The siRNA molecule (LC20Md8) and peptide (PN602) shown in Example 4 is used to form the complex of the instant example. The siRNA and peptide were complexed at incubated for six hours at room temperature and compared to a “fresh” (little to no incubation prior to anlysis) and then analyzed by gel electrophoresis.

Polyacrylamide gel electrophoresis (15% TBE Urea PAGE) and ethidium bromide staining were used to characterize the effect of the pH shift treatment on the siRNA/peptide complex. A sample of the siRNA alone (lane 1), the peptide alone (lane 2), the pre-dialzyed siRNA/peptide complex in 1×PBS and 1.5 M NaCl, the siRNA/peptide complex dialyzed against 1×PBS, the siRNA/peptide complex dialyzed against 0.1×PBS, the “fresh” siRNA/peptide complex and the “hold time” siRNA/peptide complex were analyzed by gel electrophoresis on a urea denaturing gel (15% TBE-Urea). The migration patterns of the samples on the polyacrylamide gels were visualized by exposing the ethidium bromide stained gels to UV light.

The migration pattern of the siRNA/peptide complexes on a 15% TBE-Urea polyacrlyamide gel after a “hold time” treatment of the complex was obtained. As expected, the siRNA alone (lane 1) migrated as a single distinct band while the peptide did not generate a band. As shown by the comparison of lanes 6 and 7, the six hour “hold time” treatment did not modify the siRNA/peptide complex.

These data showed that that a “hold time” treatment of the siRNA/peptide complex did not modify the complex as evidenced by a similar banding pattern to the control “fresh” complex on the gel.

### Example 9

### Modification of the siRNA/Peptide Complex Improves siRNA Mediated Gene Expression Knockdown

The present example demonstrates that modification of the siRNA/peptide complex, for example by the freeze/thaw method (F/T), thermal method (heating/cooling) and/or dialysis method, improves the in vitro efficacy of gene expression knockdown activity as mediated by the siRNA over that of the non-modified siRNA/peptide complex. The target of gene expression knockdown is the human TNF-alpha gene (hTNF-α). The significance of targeting the hTNF-α gene is that it is implicated in mediating the occurrence or progression of rheumatoid arthritis (RA) when over-expressed in human and other mammalian subjects. Therefore, targeted reduction of hTNF-α gene expression can be used as a treatment for RA.

The siRNA/Peptide complex concentrates were processed by physical and chemical means to produce putative thermodynamically stabilized forms. These forms were then diluted to either 100 or 20 nM to determine the efficacy of each formulation treatment by in vitro knock-down in isolated murine monocytes. The siRNA and peptide stock and complex samples were generated as follows: All materials (siRNA and peptides) were diluted to 1.0 mg/mL in water, pH neutralized to near 7. Molarities of each solution were calculated using the theoretical extinction coefficient for each API component. Resulting molarities for each API solution at 1.0 mg/mL are as follows: Inm4 at 75 μM; Qneg at 76 μM; PN73 at 236 μM; PN₆O₂ (an acetylated form of PN73) at 234 μM and PN826 at 233 μM. The amino acid sequence of PN826 is peptide PN73 whereby the 14th amino acid, aspartate (D), of PN73 is substituted with glutamate (E).

The treatments were divided into four groups and then those groups were sub-divided based on the peptide used and the molar ratio of the peptide to siRNA. The four treatment groups were as follows: “Mixing Only” which is a complex solution made just prior to testing; “Heating then Cooling” (thermal method) which is a slow heating at a rate of 1 degree per minute to 55° C., a hold time at 55° C. for 10 minutes, then a slow cooling back to room temperature; “Freeze-Thaw” (F/T method) which is a complex solution frozen and thawed twice (30 minutes at −80° C. and also at room temperature to ensure complete temperature transition; and the final process group and “Dialysis” where a complex solution with 1.5 M NaCl (final concentration) is dialyzed against 1×PBS solution, pH 7.2 for 4 hours.

All solutions were made as a concentrate and the concentrations are relative to the final siRNA molar concentration. A three-fold concentrate was needed to test for in vitro knock-down for each formulation; in vitro testing is done in triplicate. Also an additional five fold “concentrates” for the “100 nM” concentration groups were made for treatment (complex processing tests). After treatment, these solutions were diluted five-fold in Opti-MEM media to create the 100 nM test groups and then diluted five-fold again in Opti-MEM to create the 20 nM test groups.

Two separate sets of “concentrates” were made, one at the 1:5 molar ratio of siRNA to peptide groups (which represents a 1.0 charge ratio) and a second tube for the 1:10 molar ratio for the higher ratio groups (1.6 charge ratio). The treatment concentrates were designated A through F. A and B are Inm4 at a 1:5 (A) or 1:10 (B) molar ratio; C and D are Inm4 at a 1:5 (C) or 1:10 (D) molar ratio; and E and F are Inm4 at either a 1:5 (E) or a 1:10 (F) molar ratio. The sample volume was 250 μL of each 1500 nM concentrate was created (three-fold concentrate for in vitro testing in triplicate and five-fold concentrate for processing: 100 nM×3×5=1500 nM or 1.5 μM). Examples are given below to illustrate.

Example 1: For “A” (which is Inm4 in a 1:5 ratio with PN073) used to make the concentrate for the “Mixing Only”, “Freeze/Thaw” and “Heating and Cooling” treatment groups the final solution volumes are below in Table 8.

TABLE 8

| Component | Name | Volume |
|---|---|---|
| siRNA | Inm4 | 5 μL |
| Peptide | PN73 | 8 μL |
| Buffer | 10× PBS | 25 μL |
| Solvent | Water | 212 μL |

Example 2: For “A” (again, which is Inm4 in a 1:5 ratio with PN073) used to make the concentrate for the “Dialysis” treatment groups the final solution volumes are below in Table 9.

TABLE 9

| Component | Name | Volume |
|---|---|---|
| siRNA | Inm4 | 5 μL |
| Peptide | PN73 | 8 μL |
| Salt | 4 M NaCl | 94.5 μL |
| Buffer | 10× PBS | 25 μL |
| Solvent | Water | 117.5 μL |

TABLE 10
Summary of siRNA/Peptide Samples
Test Groups (20 nM)

| | |
|---|---|
| 1. | PN73:Inm4 (5:1) Freeze-Thaw |
| 2. | PN73:Inm4 (5:1) Heating-Cool |
| 3. | PN73:Inm4 (5:1) Salt Dialysis |
| 4. | PN73:Inm4 (5:1) Mixing only |
| 5. | PN73:Inm4 (10:1) Freeze-Thaw |
| 6. | PN73:Inm4 (10:1) Heating-Cool |
| 7. | PN73:Inm4 (10:1) Salt Dialysis |
| 8. | PN73:Inm4 (10:1) Mixing only |
| 9. | PN602:Inm4 (5:1) Freeze-Thaw |
| 10. | PN602:Inm4 (5:1) Heating-Cool |
| 11. | PN602:Inm4 (5:1) Salt Dialysis |
| 12. | PN602:Inm4 (5:1) Mixing only |
| 13. | PN602:Inm4 (10:1) Freeze-Thaw |
| 14. | PN602:Inm4 (10:1) Heating-Cool |
| 15. | PN602:Inm4 (10:1) Salt Dialysis |
| 16. | PN602:Inm4 (10:1) Mixing only |
| 17. | PN826:Inm4 (5:1) Freeze-Thaw |
| 18. | PN826:Inm4 (5:1) Heating-Cool |
| 19. | PN826:Inm4 (5:1) Salt Dialysis |
| 20. | PN826:Inm4 (5:1) Mixing only |
| 21. | PN826:Inm4 (10:1) Freeze-Thaw |
| 22. | PN826:Inm4 (10:1) Heating-Cool |
| 23. | PN826:Inm4 (10:1) Salt Dialysis |
| 24. | PN826:Inm4 (10:1) Mixing only |
| 25. | PN73:Inm4 (5:1) Freeze-Thaw |
| 26. | PN73:Inm4 (5:1) Heating-Cool |
| 27. | PN73:Inm4 (5:1) Salt Dialysis |
| 28. | PN73:Inm4 (5:1) Mixing only |
| 29. | PN73:Inm4 (10:1) Freeze-Thaw |
| 30. | PN73:Inm4 (10:1) Heating-Cool |
| 31. | PN73:Inm4 (10:1) Salt Dialysis |
| 32. | PN73:Inm4 (10:1) Mixing only |
| 33. | PN602:Inm4 (5:1) Freeze-Thaw |
| 34. | PN602:Inm4 (5:1) Heating-Cool |
| 35. | PN602:Inm4 (5:1) Salt Dialysis |
| 36. | PN602:Inm4 (5:1) Mixing only |
| 37. | PN602:Inm4 (10:1) Freeze-Thaw |
| 38. | PN602:Inm4 (10:1) Heating-Cool |
| 39. | PN602:Inm4 (10:1) Salt Dialysis |
| 40. | PN602:Inm4 (10:1) Mixing only |
| 41. | PN826:Inm4 (5:1) Freeze-Thaw |
| 42. | PN826:Inm4 (5:1) Heating-Cool |
| 43. | PN826:Inm4 (5:1) Salt Dialysis |
| 44. | PN826:Inm4 (5:1) Mixing only |
| 45. | PN826:Inm4 (10:1) Freeze-Thaw |
| 46. | PN826:Inm4 (10:1) Heating-Cool |
| 47. | PN826:Inm4 (10:1) Salt Dialysis |
| 48. | PN826:Inm4 (10:1) Mixing only |
| 49. | Inm4 #3 20 nM/lipofectamine (positive control) |
| 50. | Inm4 #3 100 nM/lipofectamine |
| 51. | Qneg 20 nM/lipofectamine (negative control) |
| 52. | Qneg 100 nM/lipofectamine (negative control) |
| 53. | Lipofectamine alone |
| 54. | Inm4 alone 20 nM |
| 55. | Inm4 alone 100 nM |
| 56. | Qneg alone 20 nM |
| 57. | Qneg alone 100 nM |
| 58. | PN73:Qneg (5:1) 100 nM |
| 59. | PN602:Qneg (5:1) 100 nM |
| 60. | PN826:Qneg (5:1) 100 nM |
| 61. | PN73 alone (5:1 dose level) at 100 nM |
| 62. | PN73 alone (10:1 dose level) at 100 nM |
| 63. | PN602 alone (5:1 dose level) at 100 nM |
| 64. | PN602 alone (10:1 dose level) at 100 nM |
| 65. | PN826 alone (5:1 dose level) at 100 nM |
| 66. | PN826 alone (10:1 dose level) at 100 nM |
| 67. | Inm4/peptide prepared by MCB |
| 68. | Qneg/peptide prepared by MCB |
| 69. | Inm4 #1 20 nM/lipofectamine (positive control) |
| 70. | Inm4 #1 100 nM/lipofectamine |
| 71. | OptiMEM (to be induced with LPS) |
| 72. | OptiMEM (not induced) |

The reagents used, including the source and grade are described below in Table 11.

TABLE 11
Materials

| Reagent | Grade | Vendor | Lot # | M.W. |
|---|---|---|---|---|
| Peptide: PN0073 | Research | Nastech | B268P158 | 4230 |
| Peptide: PN0602 | Research | Polypeptides | B318P157 | 4230 |
| Peptide: PN0826 | Research | Polypeptides | B318P160-2 | 4244 |
| siRNA: Inm4 | Research | Qiagen | B32P164 | 14274 |
| siRNA: Qneg | Research | Qiagen | B32P167 | 14195 |
| 10× PBS Concentrate | Research | Nastech | n/a | n/a |
| OptiMEM I | TC | Gibco | 1262106 | n/a |
| Hypure Water | TC | Cellgro | AQE23759 | 18 |
| Slide-A-Lyzer 2000 | Research | Pierce | GI99825 | n/a |
| MWCO | | | | |

Qneg represents a random siRNA sequence and functioned as the negative control. The levels of TNF-α mRNA were analyzed by a bDNA assay.

Table 12 shows the results of the total reduction in TNF-α mRNA in mouse monocytes dosed at 20 nM Inm4 siRNA categorized by peptide.

TABLE 12
Reduction in TNF-α mRNA in Mouse
Monocytes Dosed at 20 nM Inm4

| Complex | Method | RLU |
|---|---|---|
| PN73:Inm-4 (5:1) | Freeze Thaw | 61.86 |
| | Heat Cool | 62.14 |
| | Dialysis | 65.47 |
| | Mix | 72.14 |
| PN73:Inm-4 (10:1) | Freeze Thaw | 72.69 |
| | Heat Cool | 78.53 |
| | Dialysis | 69.64 |
| | Mix | 66.31 |
| PN602:Inm-4 (5:1) | Freeze Thaw | 70.19 |
| | Heat Cool | 68.81 |
| | Dialysis | 71.03 |
| | Mix | 75.75 |
| PN602:Inm-4 (10:1) | Freeze Thaw | 79.92 |
| | Heat Cool | 76.03 |
| | Dialysis | 78.25 |
| | Mix | 85.19 |
| PN826:Inm-4 (5:1) | Freeze Thaw | 76.58 |
| | Heat Cool | 91.31 |
| | Dialysis | 63.15 |
| | Mix | 59.05 |
| PN826:Inm-4 (10:1) | Freeze Thaw | 56.74 |
| | Heat Cool | 64.44 |
| | Dialysis | 62.64 |
| | Mix | 76.23 |
| lipo2000 | Inm-4 #1 | 21.23 |
| | Inm-4 #3 | 16.62 |
| | Qneg | 31.74 |
| | no siRNA | 58.21 |
| siRNA only | Inm-4 #3 | 53.33 |
| | Qneg | 55.64 |
| MCB PN73 5:1 | Inm-4 | 74.62 |
| | Qneg | 76.67 |
| Controls | PN73:Qneg | 59.74 |
| | PN602:Qneg | 72.31 |
| | PN826:Qneg | 64.10 |
| | cells induced | 69.64 |

A smaller RLU value indicates a greater reduction in TNF-α mRNA levels and thus a greater knockdown activity. Relative to the controls (lipofectamine and un-treated siRNA/peptide complexes), the over-all trend with the treated siRNA/peptide complexes was that the treatment reduced the level of TNF-α mRNA in cultured mouse monocytes. The results show that an overall net reduction in TNF-α mRNA in mouse monocytes dosed at 20 nM Inm4 siRNA was achieved with the heating/cooling and the F/T (freeze/thaw) method when compared to mixing alone.

The results for the total reduction in TNF-α mRNA in mouse monocytes dosed at 100 nM Inm4 siRNA are shown in Table 13.

TABLE 13
Reduction in TNF-α mRNA in Mouse
Monocytes Dosed at 20 nM Inm4

| Complex | Method | RLU |
|---|---|---|
| PN73:Inm-4 (5:1) | Freeze Thaw | 79.31 |
| | Heat Cool | 74.69 |
| | Dialysis | 62.38 |
| | Mix | 79.31 |
| PN73:Inm-4 (10:1) | Freeze Thaw | 70.59 |
| | Heat Cool | 80.33 |
| | Dialysis | 79.82 |
| | Mix | 73.92 |
| PN602:Inm-4 (5:1) | Freeze Thaw | 70.59 |
| | Heat Cool | 75.72 |
| | Dialysis | 71.10 |
| | Mix | 84.44 |
| PN602:Inm-4 (10:1) | Freeze Thaw | 90.46 |
| | Heat Cool | 74.82 |
| | Dialysis | 71.49 |
| | Mix | 71.49 |
| PN826:Inm-4 (5:1) | Freeze Thaw | 76.10 |
| | Heat Cool | 89.44 |
| | Dialysis | 67.90 |
| | Mix | 73.79 |
| PN826:Inm-4 (10:1) | Freeze Thaw | 66.87 |
| | Heat Cool | 63.79 |
| | Dialysis | 71.74 |
| | Mix | 80.72 |
| lipo2000 | Inm-4 #1 | 18.67 |
| | Inm-4 #3 | 17.64 |
| | Qneg | 37.64 |
| | no siRNA | 58.21 |
| siRNA only | Inm-4 #3 | 55.64 |
| | Qneg | 52.31 |
| Controls | PN73:Qneg | 59.74 |
| | PN602:Qneg | 72.31 |
| | PN826:Qneg | 64.10 |
| | cells induced | 69.64 |

Relative to the controls (lipofectamine and un-treated siRNA/peptide complexes), the over-all trend with the treated siRNA/peptide complexes was that the treatment reduced the level of TNF-α mRNA in cultured mouse monocytes.

Table 14 shows the overall averaging of the various treatments on TNF-α mRNA knockdown when average across all peptides and siRNA concentrations and ratios. A lower relative knockdown level indicated a lower level of TNF-α mRNA and thus a greater knockdown activity.

TABLE 14
Increase in Knockdown Activity Compared to Mixing Alone

| | Relative | |
|---|---|---|
| Method | Knockdown Level | % Increase |
| Freeze Thaw | 4.61 | 19 |
| Heat Cool | 4.66 | 18 |
| Dialysis | 5.01 | 12 |
| Mix | 5.68 | — |

There was 19% increase in knockdown activity compared to mixing alone for the freeze-thaw treatment and 18% increase for the heating-cooling cycle. The use of freeze-thaw and heat-cool treatments modifies the complexes to enhance the gene expression knockdown activity of the siRNA of the complex within a cell.

What is claimed is:

1. A complex of a double stranded (ds) ribonucleic acid and a peptide produced by the method comprising:
a. dissolving the ribonucleic acid in a first aqueous solution;
b. dissolving the peptide in a second aqueous solution;
c. mixing the first and second aqueous solutions to form a third aqueous solution; and
d. treating the third aqueous solution with one or more freezing and thawing cycles, wherein in each freezing and thawing cycle the temperature of the third aqueous solution is lowered to about −80° C. for at least 30 minutes, and subsequently increased to room temperature, thereby reducing the amount of aggregate particles of the complex in the third aqueous solution to less than ten percent of the total weight of the complex.

2. The complex of claim 1, wherein step (d) increases the molecular size of the complex.

3. The complex of claim 1, wherein the double stranded (ds) ribonucleic acid is a siRNA having 29-50 base pairs and a sequence complementary to a region of a TNF-alpha gene.

4. The complex of claim 1, wherein the double stranded (ds) ribonucleic acid is LC20.

5. The complex of claim 1, wherein the peptide is a polynucleotide delivery-enhancing polypeptide.

6. The complex of claim 1, wherein the peptide is a histone protein, or a polypeptide or peptide fragment, derivative, analog, or conjugate thereof.

7. The complex of claim 1, wherein the peptide is a polynucleotide delivery-enhancing polypeptide having an amphipathic amino acid sequence.

8. The complex of claim 1, wherein the peptide is a polynucleotide delivery-enhancing polypeptide containing a protein transduction domain or motif.

9. The complex of claim 1, wherein the peptide is a polynucleotide delivery-enhancing polypeptide containing a fusogenic peptide domain or motif.

10. The complex of claim 1, wherein the peptide is a polynucleotide delivery-enhancing polypeptide containing a ribonucleic acid-binding domain or motif and the peptide binds the ds ribonucleic acid with a Kd less than about 100 nM.

11. The complex of claim 1, wherein the peptide is selected from the group consisting of:
(SEQ ID NO: 34)
GRKKRRQRRRPPQC
(SEQ ID NO: 35)
Maleimide-AAVALLPAVLLALLAPRKKRRQRRRPPQ-amide
(SEQ ID NO: 36)
AAVALLPAVLLALLAPRKKRRQRRRPPQC
(SEQ ID NO: 37)
Maleimide-AAVALLPAVLLALLAPRKKRRQRRRPPQ-amide
(SEQ ID NO: 38)
NH2-RKKRRQRRRPPQCAAVALLPAVLLALLAP-amide
(SEQ ID NO: 39)
BrAc-GRKKRRQRRRPQ-amide
(SEQ ID NO: 40)
BrAc-RRRQRRKRGGDIMGEWGNEIFGAIAGFLG-amide
(SEQ ID NO: 41)
NH2-RRRQRRKRGGDIMGEWGNEIFGAIAGFLG-amide
(SEQ ID NO: 42)
CYGRKKRRQRRRGYGRKKRRQRRRG
(SEQ ID NO: 43)
Maleimide-GRKKRRQRRRPPQ-amide
(SEQ ID NO: 44)
NH2-KLWKAWPKLWKKLWKP-amide
(SEQ ID NO: 45)
AAVALLPAVLLALLAPRRRRRR-amide
(SEQ ID NO: 46)
RLWRALPRVLRRLLRP-amide
(SEQ ID NO: 47)
NH2-AAVALLPAVLLALLAPSGASGLDKRDYV-amide
(SEQ ID NO: 48)
Maleimide-AAVALLPAVLLALLAPSGASGLDKRDYV-amide
(SEQ ID NO: 49)
NH2-SGASGLDKRDYVAAVAALLPAVLLALLAP-amide
(SEQ ID NO: 50)
NH2-LLETLLKPFQCRICMRNFSTRQARRNHRRRHRR-amide
(SEQ ID NO: 51)
NH2-AAVACRICMRNFSTRQARRNHRRRHRR-amide
(SEQ ID NO: 52)
Maleimide-RQIKIWFQNRRMKWKK-amide
(SEQ ID NO: 53)
RQIKIWFQNRRMKWKK-amide
(SEQ ID NO: 54)
NH2-RQIKIWFQNRRMKWKKDIMGEWGNEIFGAIAGFLG-amide
(SEQ ID NO: 55)
Maleimide-SGRGKQGGKARAKAKTRSSRAGLQFPVGRVHRLLRKG-
amide
(SEQ ID NO: 56)
SGRGKQGGKARAKAKTRSSRAGLQFPVGRVHRLLRKGC-amide
(SEQ ID NO: 57)
KGSKKAVTKAQKKDGKKRKRSRK-amide
(SEQ ID NO: 58)
NH2-KKDGKKRKRSRKESYSVYVYKVLKQ-amide
(SEQ ID NO: 59)
KGSKKAVTKAQKKDGKKRKRSRKESYSVYVYKVLKQ
(SEQ ID NO: 60)
BrAc-GWTLNSAGYLLGKINLKALAALAKKIL-amide
(SEQ ID NO: 61)
KLALKLALKALKAALKLA-amide
(SEQ ID NO: 62)
BrAc-KLALKLALKALKAALKLA-amide
(SEQ ID NO: 63)
Ac-KETWWETWWTEWSQPKKKRKV-amide
(SEQ ID NO: 64)
NH2-KETWWETWWTEWSQPGRKKRRQRRRPPQ-amide
(SEQ ID NO: 65)
BrAc-RRRRRRR
(SEQ ID NO: 66)
Q|Q|Q|Q|Q|

(SEQ ID NO: 67)
NH2-RRRQRRKRGGqQqQqQqQqQ-amide
(SEQ ID NO: 68)
RVIRWFQNKRCKDKK-amide
(SEQ ID NO: 69)
Ac-LGLLLRHLRHHSNLLANI-amide
(SEQ ID NO: 70)
GQMSEIEAKVRTVKLARS-amide
(SEQ ID NO: 71)
NH2-KLWSAWPSLWSSLWKP-amide
(SEQ ID NO: 72)
NH2-KKKKKKKKK-amide
(SEQ ID NO: 73)
NH2-AARLHRFKNKGKDSTEMRRRR-amide
(SEQ ID NO: 74)
Maleimide-GLGSLLKKAGKKLKQPKSKRKV-amide
(SEQ ID NO: 75)
Maleimide-Dmt-r-FK-amide
(SEQ ID NO: 76)
Maleimide-Dmt-r-FKQqQqQqQqQq-amide
(SEQ ID NO: 77)
Maleimide-WRFK-amide
(SEQ ID NO: 78)
Maleimide-WRFKQqQqQqQqQq-amide
(SEQ ID NO: 79)
Maleimido-YRFK-amide
(SEQ ID NO: 80)
Maleimide-YRFKYRFKYRFK-amide
(SEQ ID NO: 81)
Maleimide-WRFK-amide
(SEQ ID NO: 82)
Maleimide-WRFKKSKRKV-amide
(SEQ ID NO: 83)
Maleimide-WRFKAAVALLPAVLLALLAP-amide
(SEQ ID NO: 84)
NH2-DiMeYrFK-amide
(SEQ ID NO: 85)
NH2-YrFK-amide
(SEQ ID NO: 86)
NH2-DiMeYRFK-amide
(SEQ ID NO: 87)
NH2-WrFK-amide
(SEQ ID NO: 88)
NH2-DiMeYrWK-amide
(SEQ ID NO: 89)
NH2-KFrDiMeY-amide
(SEQ ID NO: 90)
Maleimide-WRFKWRFK-amide
and
(SEQ ID NO: 91)
Maleimide-WRFKWRFKWRFK-amide.

12. The complex of claim 1, wherein the peptide is selected from the group consisting of histone H1 or a fragment thereof, histone H₂B or a fragment thereof, histone H3 or a fragment thereof, histone H4 or a fragment thereof, GKINLKALAALAKKIL(SEQ
(SEQ ID NO: 92)
GKINLKALAALAKKIL,
(SEQ ID NO: 93)
RVIRVWFQNKRCKDKK,
(SEQ ID NO: 94)
GRKKRRQRRRPPQGRKKRRQRRRPPQGRKKRRQRRRPPQ,
(SEQ ID NO: 95)
GEQIAQLIAGYIDIILKKKKSK,
WWETWKPFQCRICMRNFSTRQARRNHRRRHR (SEQ ID NO: 96), Poly Lys-Trp (4:1, MW 20,000-50,000), Poly Om-Trp (4:1, MW 20,000-50,000), and mellitin.

13. The complex of claim 1, wherein the peptide is PN73,
(SEQ ID NO: 34)
KGSKKAVTKAQKKDGKKRKRSRKESYSVYVYKVLKQ.

14. A complex of a double stranded (ds) ribonucleic acid and a peptide produced by the method comprising:
a. solubilizing the ribonucleic acid in a first aqueous solution;
b. solubilizing the peptide in a second aqueous solution;
c. mixing the solubilized ds nucleic acid and the solubilized peptide; and
d. treating the mixture with one or more heating and cooling cycles, wherein in each heating and cooling cycle the temperature of the mixture is raised to about 55° C. for at least 30 minutes, and subsequently decreased to room temperature at approximately 1° C./minute, thereby reducing the amount of aggregate particles of the complex in the third aqueous solution to less than ten percent of the total weight of the complex.

15. The complex of claim 14, wherein step (d) increases the molecular size of the complex.

16. The complex of claim 14, wherein the double stranded (ds) ribonucleic acid is a siRNA having 29-50 base pairs and a sequence complementary to a region of a TNF-alpha gene.

17. The complex of claim 14, wherein the peptide is a polynucleotide delivery-enhancing polypeptide.

18. A complex of a double stranded (ds) ribonucleic acid and a peptide produced by the method comprising:
a. dissolving the ribonucleic acid in a first aqueous solution;
b. dissolving the peptide in a second aqueous solution;
c. mixing aliquots of the first and second aqueous solutions to form a third aqueous solution;
d. raising the salt concentration of the third aqueous solution to at least 1.5 M; and
e. dialyzing the third aqueous solution to lower the salt concentration, thereby reducing the amount of aggregate particles of the complex in the third aqueous solution to less than ten percent of the total weight of the complex.

19. The complex of claim 18, wherein step (d) increases the molecular size of the complex.

20. The complex of claim 18, wherein the double stranded (ds) ribonucleic acid is a siRNA having 29-50 base pairs and a sequence complementary to a region of a TNF-alpha gene.

21. The complex of claim 18, wherein the peptide is a polynucleotide delivery-enhancing polypeptide.


SEQUENCE LISTING (100 sequences)

SEQ ID NO: 1 - protein, 7 aa, Artificial Sequence
  Description of Artificial Sequence Synthetic peptide
KRRQRRR

SEQ ID NO: 2 - protein, 16 aa, Artificial Sequence
  Description of Artificial Sequence Synthetic peptide
RQIKIWFQNRRMKWKK

SEQ ID NO: 3 - protein, 34 aa, Artificial Sequence
  Description of Artificial Sequence Synthetic peptide
DAATATRGRSAASRPTERPRAPARSASRPRRPVD

SEQ ID NO: 4 - protein, 16 aa, Artificial Sequence
  Description of Artificial Sequence Synthetic peptide
AAVALLPAVLLALLAP

SEQ ID NO: 5 - protein, 16 aa, Artificial Sequence
  Description of Artificial Sequence Synthetic peptide
AAVLLPVLLPVLLAAP

SEQ ID NO: 6 - protein, 15 aa, Artificial Sequence
  Description of Artificial Sequence Synthetic peptide
VTVLALGALAGVGVG

SEQ ID NO: 7 - protein, 17 aa, Artificial Sequence
  Description of Artificial Sequence Synthetic peptide
GALFLGWLGAAGSTMGA

SEQ ID NO: 8 - protein, 17 aa, Artificial Sequence
  Description of Artificial Sequence Synthetic peptide
MGLGLHLLVLAAALQGA

SEQ ID NO: 9 - protein, 24 aa, Artificial Sequence
  Description of Artificial Sequence Synthetic peptide
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 16 aa, Artificial Sequence
  Description of Artificial Sequence Synthetic peptide
GLFGAIAGFIENGWEG

SEQ ID NO: 15 - protein, 16 aa, Artificial Sequence
  Description of Artificial Sequence Synthetic peptide
FFGAVIGTIALGVATA

SEQ ID NO: 16 - protein, 16 aa, Artificial Sequence
  Description of Artificial Sequence Synthetic peptide
FLGFLLGVGSAIASGV

SEQ ID NO: 17 - protein, 16 aa, Artificial Sequence
  Description of Artificial Sequence Synthetic peptide
GVFVLGFLGFLATAGS

SEQ ID NO: 18 - protein, 16 aa, Artificial Sequence
  Description of Artificial Sequence Synthetic peptide
GAAIGLAWIPYFGPAA

SEQ ID NO: 19 - protein, 56 aa, Artificial Sequence
  Description of Artificial Sequence Synthetic peptide
ACTCPYCKDSEGRGSGDPGKKKQHICHIQGCGKVYGKTSHLRAHLRWHTGERPFMC

SEQ ID NO: 20 - protein, 54 aa, Artificial Sequence
  Description of Artificial Sequence Synthetic peptide
ACTCPNCKDGEKRSGEQGKKKHVCHIPDCGKTFRKTSLLRAHVRLHTGERPFVC

SEQ ID NO: 21 - protein, 55 aa, Artificial Sequence
  Description of Artificial Sequence Synthetic peptide
ACTCPNCKEGGGRGTNLGKKKQHICHIPGCGKVYGKTSHLRAHLRWHSGERPFVC

SEQ ID NO: 22 - protein, 57 aa, Artificial Sequence
  Description of Artificial Sequence Synthetic peptide
ACSCPNCREGEGRGSNEPGKKKQHICHIEGCGKVYGKTSHLRAHILRWHTGERPFIC

SEQ ID NO: 23 - protein, 60 aa, Artificial Sequence
  Description of Artificial Sequence Synthetic peptide
RCTCPNCTNEMSGLPPIVGPDERGRKQHICHIPGCERLYGKASHLKTHLRWHTGERPFLC

SEQ ID NO: 24 - protein, 58 aa, Artificial Sequence
  Description of Artificial Sequence Synthetic peptide
TCDCPNCQEAERLGPAGVHLRKKNIHSCHIPGCGKVYGKTSHLKAHLRWHTGERPFVC

SEQ ID NO: 25 - protein, 53 aa, Artificial Sequence
  Description of Artificial Sequence Synthetic peptide
RCTCPNCKAIKHGDRGSQHTHLCSVPGCGKTYKKTSHLRAHLRKHTGDRPFVC

SEQ ID NO: 26 - protein, 56 aa, Artificial Sequence
  Description of Artificial Sequence Synthetic peptide
PQISLKKKIFFFIFSNFRGDGKSRIHICHLCNKTYGKTSHLRAHLRGHAGNKPFAC

SEQ ID NO: 27 - protein, 31 aa, Artificial Sequence
  Description of Artificial Sequence Synthetic peptide
WWETWKPFQCRICMRNFSTRQARRNHRRRHR

SEQ ID NO: 28 - protein, 16 aa, Artificial Sequence
  Description of Artificial Sequence Synthetic peptide
GKINLKALAALAKKIL

SEQ ID NO: 29 - protein, 16 aa, Artificial Sequence
  Description of Artificial Sequence Synthetic peptide
RVIRVWFQNKRCKDKK

SEQ ID NO: 30 - protein, 39 aa, Artificial Sequence
  Description of Artificial Sequence Synthetic peptide
GRKKRRQRRRPPQGRKKRRQRRRPPQGRKKRRQRRRPPQ

SEQ ID NO: 31 - protein, 22 aa, Artificial Sequence
  Description of Artificial Sequence Synthetic peptide
GEQIAQLIAGYIDIILKKKKSK

SEQ ID NO: 32 - DNA, 21 bp, Artificial Sequence
  Description of Combined DNA/RNA Molecule Synthetic oligonucleotide
  Description of Artificial Sequence Synthetic oligonucleotide
gggucggaacccaagcuuatt

SEQ ID NO: 33 - DNA, 21 bp, Artificial Sequence
  Description of Combined DNA/RNA Molecule Synthetic oligonucleotide
  Description of Artificial Sequence Synthetic oligonucleotide
atcccagccuuggguucgaau

SEQ ID NO: 34 - protein, 14 aa, Artificial Sequence
  Description of Artificial Sequence Synthetic peptide
GRKKRRQRRRPPQC

SEQ ID NO: 35 - protein, 28 aa, Artificial Sequence
  Description of Artificial Sequence Synthetic peptide
  c-term amidated
  MOD_RES (1): Maleimide-Ala
AAVALLPAVLLALLAPRKKRRQRRRPPQ

SEQ ID NO: 36 - protein, 29 aa, Artificial Sequence
  Description of Artificial Sequence Synthetic peptide
AAVALLPAVLLALLAPRKKRRQRRRPPQC

SEQ ID NO: 37 - protein, 28 aa, Artificial Sequence
  Description of Artificial Sequence Synthetic peptide
  c-term amidated
  MOD_RES (1): Maleimide-Ala
AAVALLPAVLLALLAPRKKRRQRRRPPQ

SEQ ID NO: 38 - protein, 29 aa, Artificial Sequence
  Description of Artificial Sequence Synthetic peptide
  c-term amidated
RKKRRQRRRPPQCAAVALLPAVLLALLAP

SEQ ID NO: 39 - protein, 12 aa, Artificial Sequence
  Description of Artificial Sequence Synthetic peptide
  c-term amidated
  MOD_RES (1): BrAc-Gly
GRKKRRQRRRPQ

SEQ ID NO: 40 - protein, 29 aa, Artificial Sequence
  Description of Artificial Sequence Synthetic peptide
  c-term amidated
  MOD_RES (1): BrAc-Arg
RRRQRRKRGGDIMGEWGNEIFGAIAGFLG

SEQ ID NO: 41 - protein, 29 aa, Artificial Sequence
  Description of Artificial Sequence Synthetic peptide
  c-term amidated
RRRQRRKRGGDIMGEWGNEIFGAIAGFLG

SEQ ID NO: 42 - protein, 25 aa, Artificial Sequence
  Description of Artificial Sequence Synthetic peptide
CYGRKKRRQRRRGYGRKKRRQRRRG

SEQ ID NO: 43 - protein, 13 aa, Artificial Sequence
  Description of Artificial Sequence Synthetic peptide
  c-term amidated
  MOD_RES (1): Maleimide-Gly
GRKKRRQRRRPPQ

SEQ ID NO: 44 - protein, 16 aa, Artificial Sequence
  Description of Artificial Sequence Synthetic peptide
  c-term amidated
KLWKAWPKLWKKLWKP

SEQ ID NO: 45 - protein, 22 aa, Artificial Sequence
  Description of Artificial Sequence Synthetic peptide
  c-term amidated
AAVALLPAVLLALLAPRRRRRR

SEQ ID NO: 46 - protein, 16 aa, Artificial Sequence
  Description of Artificial Sequence Synthetic peptide
  c-term amidated
RLWRALPRVLRRLLRP

SEQ ID NO: 47 - protein, 28 aa, Artificial Sequence
  Description of Artificial Sequence Synthetic peptide
  c-term amidated
AAVALLPAVLLALLAPSGASGLDKRDYV

SEQ ID NO: 48 - protein, 28 aa, Artificial Sequence
  Description of Artificial Sequence Synthetic peptide
  c-term amidated
  MOD_RES (1): Maleimide-Ala
AAVALLPAVLLALLAPSGASGLDKRDYV

SEQ ID NO: 49 - protein, 29 aa, Artificial Sequence
  Description of Artificial Sequence Synthetic peptide
  c-term amidated
SGASGLDKRDYVAAVAALLPAVLLALLAP

SEQ ID NO: 50 - protein, 33 aa, Artificial Sequence
  Description of Artificial Sequence Synthetic peptide
  c-term amidated
LLETLLKPFQCRICMRNFSTRQARRNHRRRHRR

SEQ ID NO: 51 - protein, 27 aa, Artificial Sequence
  Description of Artificial Sequence Synthetic peptide
  c-term amidated
AAVACRICMRNFSTRQARRNHRRRHRR

SEQ ID NO: 52 - protein, 16 aa, Artificial Sequence
  Description of Artificial Sequence Synthetic peptide
  c-term amidated
  MOD_RES (1): Maleimide-Arg
RQIKIWFQNRRMKWKK

SEQ ID NO: 53 - protein, 16 aa, Artificial Sequence
  Description of Artificial Sequence Synthetic peptide
  c-term amidated
RQIKIWFQNRRMKWKK

SEQ ID NO: 54 - protein, 35 aa, Artificial Sequence
  Description of Artificial Sequence Synthetic peptide
  c-term amidated
RQIKIWFQNRRMKWKKDIMGEWGNEIFGAIAGFLG

SEQ ID NO: 55 - protein, 37 aa, Artificial Sequence
  Description of Artificial Sequence Synthetic peptide
  c-term amidated
  MOD_RES (1): Maleimide-Ser
SGRGKQGGKARAKAKTRSSRAGLQFPVGRVHRLLRKG

SEQ ID NO: 56 - protein, 38 aa, Artificial Sequence
  Description of Artificial Sequence Synthetic peptide
  c-term amidated
SGRGKQGGKARAKAKTRSSRAGLQFPVGRVHRLLRKGC

SEQ ID NO: 57 - protein, 23 aa, Artificial Sequence
  Description of Artificial Sequence Synthetic peptide
  c-term amidated
KGSKKAVTKAQKKDGKKRKRSRK

SEQ ID NO: 58 - protein, 25 aa, Artificial Sequence
  Description of Artificial Sequence Synthetic peptide
  c-term amidated
KKDGKKRKRSRKESYSVYVYKVLKQ

SEQ ID NO: 59 - protein, 36 aa, Artificial Sequence
  Description of Artificial Sequence Synthetic peptide
KGSKKAVTKAQKKDGKKRKRSRKESYSVYVYKVLKQ

SEQ ID NO: 60 - protein, 27 aa, Artificial Sequence
  Description of Artificial Sequence Synthetic peptide
  c-term amidated
  MOD_RES (1): BrAc-Gly
GWTLNSAGYLLGKINLKALAALAKKIL

SEQ ID NO: 61 - protein, 18 aa, Artificial Sequence
  Description of Artificial Sequence Synthetic peptide
  c-term amidated
KLALKLALKALKAALKLA

SEQ ID NO: 62 - protein, 18 aa, Artificial Sequence
  Description of Artificial Sequence Synthetic peptide
  c-term amidated
  MOD_RES (1): BrAc-Lys
KLALKLALKALKAALKLA

SEQ ID NO: 63 - protein, 21 aa, Artificial Sequence
  Description of Artificial Sequence Synthetic peptide
  n-term acylated
  c-term amidated
KETWWETWWTEWSQPKKKRKV

SEQ ID NO: 64 - protein, 28 aa, Artificial Sequence
  Description of Artificial Sequence Synthetic peptide
  c-term amidated
KETWWETWWTEWSQPGRKKRRQRRRPPQ

SEQ ID NO: 65 - protein, 7 aa, Artificial Sequence
  Description of Artificial Sequence Synthetic peptide
  MOD_RES (1): BrAc-Arg
RRRRRRR

SEQ ID NO: 66 - protein, 10 aa, Artificial Sequence
  Description of Artificial Sequence Synthetic peptide
  MOD_RES (2): D-Gln
  MOD_RES (4): D-Gln
  MOD_RES (6): D-Gln
  MOD_RES (8): D-Gln
  MOD_RES (10): D-Gln
QQQQQQQQQQ

SEQ ID NO: 67 - protein, 20 aa, Artificial Sequence
  Description of Artificial Sequence Synthetic peptide
  c-term amidated
  MOD_RES (11): D-Gln
  MOD_RES (13): D-Gln
  MOD_RES (15): D-Gln
  MOD_RES (17): D-Gln
  MOD_RES (19): D-Gln
RRRQRRKRGGQQQQQQQQQQ

SEQ ID NO: 68 - protein, 15 aa, Artificial Sequence
  Description of Artificial Sequence Synthetic peptide
  c-term amidated
RVIRWFQNKRCKDKK

SEQ ID NO: 69 - protein, 18 aa, Artificial Sequence
  Description of Artificial Sequence Synthetic peptide
  n-term acylated
  c-term amidated
LGLLLRHLRHHSNLLANI

SEQ ID NO: 70 - protein, 18 aa, Artificial Sequence
  Description of Artificial Sequence Synthetic peptide
GQMSEIEAKVRTVKLARS

SEQ ID NO: 71 - protein, 16 aa, Artificial Sequence
  Description of Artificial Sequence Synthetic peptide
KLWSAWPSLWSSLWKP

SEQ ID NO: 72 - protein, 9 aa, Artificial Sequence
  Description of Artificial Sequence Synthetic peptide
  c-term amidated
KKKKKKKKK

SEQ ID NO: 73 - protein, 21 aa, Artificial Sequence
  Description of Artificial Sequence Synthetic peptide
  c-term amidated
AARLHRFKNKGKDSTEMRRRR

SEQ ID NO: 74 - protein, 22 aa, Artificial Sequence
  Description of Artificial Sequence Synthetic peptide
  c-term amidated
  MOD_RES (1): Maleimide-Gly
GLGSLLKKAGKKLKQPKSKRKV

SEQ ID NO: 75 - protein, 4 aa, Artificial Sequence
  Description of Artificial Sequence Synthetic peptide
  c-term amidated
  MOD_RES (1): Maleimide-Dmt
  MOD_RES (2): D-Arg
XRFK

SEQ ID NO: 76 - protein, 14 aa, Artificial Sequence
  Description of Artificial Sequence Synthetic peptide
  c-term amidated
  MOD_RES (1): Maleimide-Dmt
  MOD_RES (6): D-Arg
  MOD_RES (8): D-Gln
  MOD_RES (10): D-Gln
  MOD_RES (12): D-Gln
  MOD_RES (14): D-Gln
XRFKQQQQQQQQQQ

SEQ ID NO: 77 - protein, 4 aa, Artificial Sequence
  Description of Artificial Sequence Synthetic peptide
  c-term amidated
  MOD_RES (1): Maleimide-Trp
WRFK

SEQ ID NO: 78 - protein, 14 aa, Artificial Sequence
  Description of Artificial Sequence Synthetic peptide
  c-term amidated
  MOD_RES (1): Maleimide-Trp
  MOD_RES (6): D-Gln
  MOD_RES (8): D-Gln
  MOD_RES (10): D-Gln
  MOD_RES (12): D-Gln
  MOD_RES (14): D-Gln
WRFKQQQQQQQQQQ

SEQ ID NO: 79 - protein, 4 aa, Artificial Sequence
  Description of Artificial Sequence Synthetic peptide
  c-term amidated
  MOD_RES (1): Maleimido-Tyr
YRFK

SEQ ID NO: 80 - protein, 12 aa, Artificial Sequence
  Description of Artificial Sequence Synthetic peptide
  c-term amidated
  MOD_RES (1): Maleimide-Tyr
YRFKYRFKYRFK

SEQ ID NO: 81 - protein, 4 aa, Artificial Sequence
  Description of Artificial Sequence Synthetic peptide
  c-term amidated
  MOD_RES (1): Maleimide-Trp
WRFK

SEQ ID NO: 82 - protein, 10 aa, Artificial Sequence
  Description of Artificial Sequence Synthetic peptide
  c-term amidated
  MOD_RES (1): Maleimide-Trp
WRFKKSKRKV

SEQ ID NO: 83 - protein, 20 aa, Artificial Sequence
  Description of Artificial Sequence Synthetic peptide
  c-term amidated
  MOD_RES (1): Maleimide-Trp
WRFKAAVALLPAVLLALLAP

SEQ ID NO: 84 - protein, 4 aa, Artificial Sequence
  Description of Artificial Sequence Synthetic peptide
  c-term amidated
  MOD_RES (1): Dmt
  MOD_RES (2): D-Arg
XRFK

SEQ ID NO: 85 - protein, 4 aa, Artificial Sequence
  Description of Artificial Sequence Synthetic peptide
  c-term amidated
  MOD_RES (2): D-Arg
YRFK

SEQ ID NO: 86 - protein, 4 aa, Artificial Sequence
  Description of Artificial Sequence Synthetic peptide
  c-term amidated
  MOD_RES (1): Dmt
XRFK

SEQ ID NO: 87 - protein, 4 aa, Artificial Sequence
  Description of Artificial Sequence Synthetic peptide
  MOD_RES (2): D-Arg
WRFK

SEQ ID NO: 88 - protein, 4 aa, Artificial Sequence
  Description of Artificial Sequence Synthetic peptide
  c-term amidated
  MOD_RES (1): Dmt
  MOD_RES (2): D-Arg
XRWK

SEQ ID NO: 89 - protein, 4 aa, Artificial Sequence
  Description of Artificial Sequence Synthetic peptide
  MOD_RES (3): D-Arg
  MOD_RES (4): Dmt
KFRX

SEQ ID NO: 90 - protein, 8 aa, Artificial Sequence
  Description of Artificial Sequence Synthetic peptide
  c-term amidated
  MOD_RES (1): Maleimide-Trp
WRFKWRFK

SEQ ID NO: 91 - protein, 12 aa, Artificial Sequence
  Description of Artificial Sequence Synthetic peptide
  c-term amidated
  MOD_RES (1): Maleimide-Trp
WRFKWRFKWRFK

SEQ ID NO: 92 - protein, 16 aa, Artificial Sequence
  Description of Artificial Sequence Synthetic peptide
GKINLKALAALAKKIL

SEQ ID NO: 93 - protein, 16 aa, Artificial Sequence
  Description of Artificial Sequence Synthetic peptide
RVIRVWFQNKRCKDKK

SEQ ID NO: 94 - protein, 39 aa, Artificial Sequence; identical to SEQ ID NO: 30
  Description of Artificial Sequence Synthetic peptide

SEQ ID NO: 95 - protein, 22 aa, Artificial Sequence
  Description of Artificial Sequence Synthetic peptide
GEQIAQLIAGYIDIILKKKKSK

SEQ ID NO: 96 - protein, 31 aa, Artificial Sequence; identical to SEQ ID NO: 27
  Description of Artificial Sequence Synthetic peptide

SEQ ID NO: 97 - protein, 23 aa, Artificial Sequence
  Description of Combined DNA/RNA Molecule Synthetic oligonucleotide
  Description of Artificial Sequence Synthetic peptide
  MOD_RES (2)..(5): region may encompass 2-4 variable amino acids
  MOD_RES (7)..(18): variable amino acid
  MOD_RES (20)..(22): variable amino acid
CXXXXCXXXXXXXXXXXXHXXXH

SEQ ID NO: 98 - DNA, 21 bp, Artificial Sequence
  Description of Artificial Sequence Synthetic oligonucleotide
  modified_base (1): 2′-O-methyl modified ribonucleotide
  modified_base (2): 2′-O-methyl modified ribonucleotide
  modified_base (4): ribothymidine
  modified_base (17)..(18): ribothymidine
gggtcggaacccaagcttatt

SEQ ID NO: 99 - DNA, 21 bp, Artificial Sequence
  Description of Artificial Sequence Synthetic oligonucleotide
  modified_base (1): 2′-O-methyl modified ribonucleotide
  modified_base (2): 2′-O-methyl modified ribonucleotide
  modified_base (6)..(7): ribothymidine
  modified_base (11)..(12): ribothymidine
uaagcttgggttccgacccta

SEQ ID NO: 100 - protein, 36 aa, Artificial Sequence; identical to SEQ ID NO: 59
  Description of Artificial Sequence Synthetic peptide